(19) United States
(12) Patent Application Publication
(10) Pub. No.: US 2024/0358668 A1
(43) Pub. Date: Oct. 31, 2024

(54) METHODS AND COMPISITIONS FOR AMELIORATING BIOMARKERS ASSOCIATED WITH CARDIOVASCUALR RISK USING (R)-2-AMINO-3-PHENYLPROPYL CARBAMATE

(71) Applicant: AXSOME THERAPEUTICS, INC., New York, NY (US)

(72) Inventor: Lawrence Patrick CARTER, Palo Alto, CA (US)

(21) Appl. No.: 18/687,962

(22) PCT Filed: Sep. 2, 2022
(86) PCT No.: PCT/US2022/075873

§ 371 Date: Feb. 29, 2024

Related U.S. Application Data
(60) Provisional application No. 63/240,422, filed on Sep. 3, 2021.

(51) Int. Cl. A61K 31/27; A61P 3/04; A61P 9/12

(52) U.S. Cl. CPC A61K 31/27; A61P 3/04; A61P 9/12

(57) ABSTRACT

The present invention provides methods for amelioration of biomarkers associated with cardiovascular risk with (R)-2-amino-3-phenylpropyl carbamate (APC).

### STATEMENT OF PRIORITY

This application claims the benefit of U.S. Provisional Application Ser. No. 63/240,422, filed Sep. 3, 2021, the entire contents of which are incorporated by reference herein.

### TECHNICAL FIELD

The present invention provides methods for ameliorating biomarkers associated with cardiovascular risk with (R)-2-amino-3-phenylpropyl carbamate (APC).

### INCORPORATION BY REFERENCE

Compositions, methods of treatment, dosing, and methods for producing APC (which also has other names) and related compounds can be found in U.S. Pat. Nos. 8,232,315; 8,440,715; 8,552,060; 8,623,913; 8,729,120; 8,741,950; 8,877,806; 8,895,609; 8,927,602; 9,226,910; 9,359,290; 9,403,761; 9,604,917; 9,610,274; 9,907,777; 10,195,151; 10,351,517; 10,512,609; and U.S. Publication Nos. 2015/0018414 and 2020/0085748 and are incorporated by reference herein for these purposes. Methods for producing APC (which also has other names) and related compounds can also be found in U.S. Pat. Nos. 5,955,499; 5,705,640; 6,140,532 and 5,756,817. All of the above patents and applications are hereby incorporated by reference in their entireties for all purposes.

### BACKGROUND

(R)-2-amino-3-phenylpropyl carbamate (APC) is a phenylalanine analog that has been demonstrated to be useful in the treatment of patients with excessive daytime sleepiness (EDS) associated with obstructive sleep apnea (OSA) and narcolepsy. Traditionally, medications which have been found useful for the treatment for these indications have resulted in increased cardiovascular risk, and warning labels have been required on therapeutics which are presently prescribed to patients for these indications.

There is a need for treatment options that do not have side effects, particularly those that ameliorate biomarkers associated with cardiovascular risk.

### BRIEF SUMMARY OF THE INVENTION

Accordingly, the present disclosure provides methods of ameliorating biomarkers associated with cardiovascular risk using (R)-2-amino-3-phenylpropyl carbamate (APC).

### BRIEF DESCRIPTION OF THE DRAWINGS

The invention may be best understood from the following detailed description when read in conjunction with the accompanying drawings. Included in the drawings are the following figures.

FIG. 1 shows ABPM vital sign data for 14-002: SBP by ABPM at Screening and Week 8 (Safety Population). Abscissa represents time point bins in hours of the day. ABPM, ambulatory blood pressure monitoring; mmHg, millimeters mercury; SBP, systolic blood pressure.

FIG. 2 shows ABPM vital sign data for 14-002: DBP by ABPM at Screening and Week 8 (Safety Population). Abscissa represents time point bins in hours of the day. ABPM, ambulatory blood pressure monitoring; mmHg, millimeters mercury; DBP, diastolic blood pressure.

FIG. 3 shows ABPM vital sign data for 14-002: HR by ABPM at Screening and Week 8 (Safety Population). Abscissa represents time point bins in hours of the day. ABPM, ambulatory blood pressure monitoring; bmp, beats per minute; HR, heart rate.

FIG. 4 shows ABPM vital sign data for 14-003: SBP by ABPM at Screening and Week 8 (Safety Population). Abscissa represents time point bins in hours of the day. ABPM, ambulatory blood pressure monitoring; mmHg, millimeters mercury; SBP, systolic blood pressure.

FIG. 5 shows ABPM vital sign data for 14-003: DBP by ABPM at Screening and Week 8 (Safety Population). Abscissa represents time point bins in hours of the day. ABPM, ambulatory blood pressure monitoring; mmHg, millimeters mercury; DBP, diastolic blood pressure.

FIG. 6 shows ABPM vital sign data for 14-003: HR by ABPM at Screening and Week 8 (Safety Population). Abscissa represents time point bins in hours of the day. ABPM, ambulatory blood pressure monitoring; bmp, beats per minute; HR, heart rate.

FIG. 7 shows change in condensed dipper categories from screening to week 8 in study 14-002 (safety population with data at both time points). Dipper is further defined as butt this dipping≥10% and <20% or extreme dipper (dipping≥20%). Percent (%) change is calculated as [(% at week 8)/(% at screening)]×100. BP is blood pressure. MAP is mean arterial pressure.

FIG. 8 shows solriamfetol treatment did not increase the percentage of participants with narcolepsy with a non-dipping BP profile. Dipper, dipping≥10%; non-dipper, dipping<10% based on changes in blood pressure from 10 p.m. to 7 a.m., representing the sleep period. BP, blood pressure; Scr, screening; Wk, week.

FIG. 9 shows change in condensed dipper categories from screening to week 8 in study 14-003 (safety population with data at both time points). Dipper is further defined as dipping≥10% and <20% or extreme dipper (dipping≥20%). Percent (%) change is calculated as [(% at week 8)/(% at screening)]×100. BP is blood pressure. MAP is mean arterial pressure.

FIG. 10 shows solriamfetol treatment did not increase the percentage of participants with OSA with a non-dipping BP profile. Dipper, dipping≥10%; non-dipper, dipping<10% based on changes in blood pressure from 10 p.m. to 7 a.m., representing the sleep period. BP, blood pressure; OSA, obstructive sleep apnea; Scr, screening; Wk, week.

### DETAILED DESCRIPTION OF THE INVENTION

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

All publications, patent applications, patents, and other references mentioned herein are incorporated by reference herein in their entirety for all purposes.

(R)-2-amino-3-phenylpropyl carbamate (APC) is a phenylalanine analog that has been demonstrated to be useful in the treatment of patients with excessive daytime sleepiness (EDS) associated with obstructive sleep apnea (OSA) and narcolepsy. The structure of APC is given below as Formula (I).

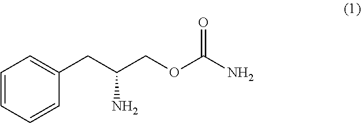 [SMILES: NC(=O)OC[C@H](N)Cc1ccccc1]

APC shows activity as a dopamine and norepinephrine reuptake inhibitor (DNRI). DNRIs have historically been used for treatment of a variety of diseases and disorders, including depression, attention deficit hyperactivity disorder (ADHD), narcolepsy, and obstructive sleep apnea (OSA). However, many DNRIs are known to have severe side effects. One common problem with DNRIs that has become apparent over the years are the effects DNRIs have on the cardiovascular system. Effects include elevated blood pressure, elevated heart rate, and hypertension (Wilens et al., Blood Pressure Changes Associated With Medication Treatment of Adults With Attention-Deficit/Hyperactivity Disorder, J. Clin. Psychiatry 2005; 66(2):253-259). Severe cardiac effects, including mortality from cardiovascular toxicity, have also been seen in certain types of use (Lidder et al., Cardiovascular Toxicity Associated with Recreational Use of Diphenylprolinol (diphenyl-2-pyrrolidinemethanol [D2PM]) Journal of Medical Toxicology, September 2008, 4(2): 167-169).

Association of DNRIs and cardiovascular risk is also disclosed in the prescribing information for some DNRI therapeutics currently on the market. For instance, the DNRI Focalin®, also known as dexmethylphenidate, discloses warnings of serious cardiovascular events in its prescribing information, and states that a modest increase in blood pressure of 2-4 mm Hg or potentially higher may result from taking the medication (see Focalin® prescribing information). Another DNRI, Wellbutrin®, also known as bupropion hydrochloride, has been used for treatment of depression and smoking cessation, however, studies with Wellbutrin® have also reported increases in biomarkers associated with cardiovascular risk, such as blood pressure and heart rate (Wilens et al., Blood Pressure Changes Associated With Medication Treatment of Adults With Attention-Deficit/Hyperactivity Disorder, J. Clin. Psychiatry 2005; 66(2):253-259). The prescribing information for Wellbutrin® has warnings indicating potentially adverse cardiovascular effects, including severe hypertension after treatment with Wellbutrin® (see Wellbutrin® prescribing information).

The prescribing information warnings indicate that physicians considering these drugs for their patients would be dissuaded from prescribing them to those patients with a biomarker profile associated with increased cardiovascular risk. In addition, physicians considering DNRIs for patients without a present cardiovascular risk would be incentivized to carefully monitor these risk factors over time while using DNRIs to ensure patient safety.

Interestingly, treatment with (R)-2-amino-3-phenylpropyl carbamate (APC) has shown improvements in various biomarkers associated with cardiovascular risk in patients after treatment. From baseline to week 40 after treatment with APC, biomarker data was collected to assess the effects of treatment with APC on the cardiovascular system. Biomarkers measured include systolic blood pressure, diastolic blood pressure, triglycerides, cholesterol, and glucose. The results show there were decreases in blood pressure and percentages of patients with lower glucose and triglycerides. These results are surprising and unexpected given that NDRIs are commonly associated with increases in biomarkers associated with cardiovascular risk. A survey conducted with clinicians demonstrated that weight loss and improvement in other CV/CM risk factors would be important and impactful to clinicians and would positively affect how they think about using APC (solriamfetol) to treat OSA patients. Clinicians reported high unmet need with regard to overweight and obesity in OSA, ease of objective diagnosis/identification of overweight patients, and high differentiation of this type of effect from other treatment options. When physicians (n=30) were queried about the influence of a weight/cardiometabolic benefit for solriamfetol on their prescribing, a majority reported robust enthusiasm.

Data provided from patients indicates that APC has a superior cardiovascular profile compared to other NDRIs.

### I. Definitions

Unless otherwise expressly defined, the terms used herein will be understood according to their ordinary meaning in the art.

Sunosi® (NDA 211230), also known as solriamfetol, contains the phenylalanine derivative solriamfetol, also known as (R)-2-amino-3-phenylpropyl carbamate (see Sunosi® package insert, incorporated herein by reference).

As defined herein, the term “biomarkers associated with cardiovascular risk” refers to a measurable substance in an organism whose presence is indicative of the presence of an increased risk of cardiovascular disease or actual cardiovascular disease. Cardiovascular disease includes but is not limited to: atherosclerosis, stroke, coronary artery disease, heart failure, abnormal heart rhythm or arrhythmias, aorta disease, Marfan syndrome, deep vein thrombosis, pulmonary embolism, heart attack, cardiomyopathy, heart valve disease, blood vessel disease, rheumatic heart disease, pericardial disease, angina, mitral regurgitation, pulmonary stenosis, mitral valve prolapse, carotid artery disease, peripheral artery disease, and hypertension. Biomarkers associated with cardiovascular risk include, without limitation, systolic blood pressure, diastolic blood pressure, triglyceride levels, cholesterol levels, and glucose levels.

As used herein, the term “about” modifying, for example, the dimensions, volumes, quantity of an ingredient in a composition, concentrations, process temperature, process time, yields, flow rates, pressures, and like values, and ranges thereof, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 10, 9, 8, 7, 6, 5, 4, 3, 2, 1 percent or less of the stated reference value.

The term “subject” or “patient” intends an animal, a mammal, or yet further a human patient.

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, etc., as would be well known in the art.

### II. (R)-2-Amino-3-Phenylpropyl Carbamate (APC) and Related Compounds

(R)-2-amino-3-phenylpropyl carbamate (APC) is a phenylalanine analog that has been demonstrated to be useful in ameliorating biomarkers associated with cardiovascular risk. The structure of APC is given below as Formula (I).

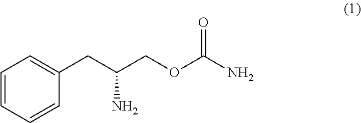 [SMILES: NC(=O)OC[C@H](N)Cc1ccccc1]

APC shows activity as a dopamine and norepinephrine reuptake inhibitor (DNRI). DNRIs have historically been used for treatment of a variety of diseases and disorders, including depression, attention deficit hyperactivity disorder (ADHD), narcolepsy, and obstructive sleep apnea (OSA).

In addition, other compounds related to APC disclosed in Formula (I) are contemplated for ameliorating biomarkers associated with cardiovascular risk. Additional compounds contemplated for ameliorating biomarkers associated with cardiovascular risk are disclosed in U.S. Pat. Nos. 8,232,315; 8,440,715; 8,552,060; 8,623,913; 8,729,120; 8,741,950; 8,877,806; 8,895,609; 8,927,602; 9,226,910; 9,359,290; 9,403,761; 9,604,917; 9,610,274; 9,907,777; 10,195,151; 10,351,517; 10,512,609; and U.S. Publication Nos. 2015/0018414 and 2020/0085748 and are incorporated by reference herein for these purposes. The term “an APC compound” as used herein refers to APC and any of the related compounds disclosed herein.

The present invention is based in part on the discovery that phenylalkylamino carbamates have novel and unique pharmacological properties. One compound of Formula (II) consists of the (D) enantiomer of the structure shown below wherein R₁═R₂=hydrogen and x is 0, in the structure shown below the amine group is directed down from the plane of the paper,

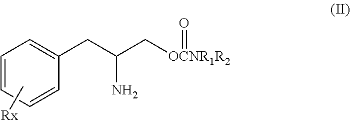

wherein R is a member selected from the group consisting of hydrogen, lower alkyl of 1 to 8 carbon atoms, halogen selected from F, Cl, Br and I, alkoxy containing 1 to 3 carbon atoms, nitro, hydroxy, trifluoromethyl, and thioalkoxy containing 1 to 3 carbon atoms; x is an integer of 1 to 3, with the proviso that R may be the same or different when x is 2 or 3; and
R₁ and R₂ can be the same or different from each other and are independently selected from the group consisting of hydrogen, lower alkyl of 1 to 8 carbon atoms, aryl, arylalkyl, and cycloalkyl of 3 to 7 carbon atoms;
or R₁ and R₂ can be joined to form a 5 to 7-membered heterocycle substituted with a member selected from the group consisting of hydrogen, alkyl, and aryl groups, wherein the cyclic compound can comprise 1 to 2 nitrogen atoms and 0 to 1 oxygen atom, wherein the nitrogen atoms are not directly connected with each other or with the oxygen atom.

This compound is the (R) enantiomer, if named by structure and is therefore (R)-(beta-amino-benzenepropyl) carbamate. This compound is the dextrorotary enantiomer and can therefore also be named O-carbamoyl-(D)-phenylalaninol and is referred to herein as the “test compound”. The two chemical names may be used interchangeably in this specification.

Thus, in some embodiments, the present invention is directed to a method of ameliorating biomarkers associated with cardiovascular risk, the method comprising administering to a subject in need thereof a therapeutically effective amount of a compound selected from the group consisting of phenylalkylamino carbamates of the following Formula (II):

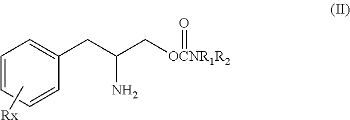

or an enantiomer, diastereomer, racemate or mixtures thereof, or a pharmaceutically acceptable salt or ester thereof wherein; R is a member selected from the group consisting of hydrogen, lower alkyl of 1 to 8 carbon atoms, halogen selected from F, Cl, Br and I, alkoxy containing 1 to 3 carbon atoms, nitro, hydroxy, trifluoromethyl, and thioalkoxy containing 1 to 3 carbon atoms; x is an integer of 1 to 3, with the proviso that R may be the same or different when x is 2 or 3; R₁ and R₂ can be the same or different from each other and are independently selected from the group consisting of hydrogen, lower alkyl of 1 to 8 carbon atoms, aryl, arylalkyl, cycloalkyl of 3 to 7 carbon atoms; or R₁ and R₂ can be joined to form a 5 to 7-membered heterocycle substituted with a member selected from the group consisting of hydrogen, alkyl, and aryl groups, wherein the cyclic compound can comprise 0 to 2 nitrogen atoms and 0 to 1 oxygen atoms, wherein the nitrogen atoms are not directly connected with each other or with the oxygen atom and the pharmaceutically acceptable salts and esters thereof.

The present method also includes the use of a compound selected from the group consisting of Formula (II) wherein R₁ and R₂ are preferably selected from hydrogen and x preferably is 0, this is Formula (IIa) below.

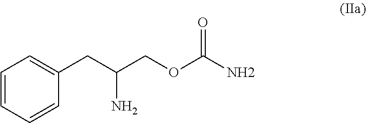 [SMILES: NC(=O)OCC(N)Cc1ccccc1]

The present method also includes the use of the D enantiomer selected from the group consisting of Formula (II) or an enantiomeric mixture wherein the D enantiomer selected from the group consisting of Formula (IIa) predominates wherein R₁ and R₂ are preferably selected from hydrogen and x preferably is 0, this is O-carbamoyl-(D)-phenylalaninol. Formula (IIb) below; (note-in Formula (IIb), i.e. the D enantiomer, as shown, the amine group on the chiral carbon is orientated into the plane of the paper)

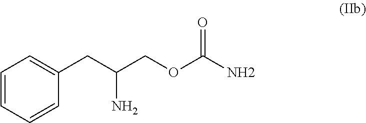 [SMILES: NC(=O)OCC(N)Cc1ccccc1]

For enantiomeric mixtures wherein one enantiomer selected from the group consisting of Formula (II) predominates, preferably, an enantiomer selected from the group consisting of Formula (II) predominates to the extent of about 90% or greater. More preferably, an enantiomer selected from the group consisting of Formula (II) predominates to the extent of about 98% or greater.

The compounds of Formula (II) can be synthesized by methods known to a skilled artisan. The salts and esters of the compounds of Formula (II) can be produced by treating the compound with a suitable mineral or organic acid (HX) in suitable solvent or by other means well known to those of skill in the art.

Details of the above reactions schemes for synthesizing compounds of Formula (II) as well as representative examples on the preparation of specific compounds have been described in U.S. Pat. Nos. 5,705,640, 5,955,499, 6,140,532, all incorporated herein by reference in their entirety.

From Formula (II) it is evident that some of the compounds of the invention have at least one and possibly more asymmetric carbon atoms. It is intended that the present invention include within its scope the stereochemically pure isomeric forms of the compounds as well as their racemates. Stereochemically pure isomeric forms may be obtained by the application of art known principles. Diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from appropriate stereochemically pure starting materials, or by using stereoselective reactions.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, Third Edition, John Wiley & Sons, 1999. The protecting groups may be removed at a convenient subsequent stage using methods known from the art.

This disclosure provides a compound represented by Formula (III) and a pharmaceutical acceptable salt:

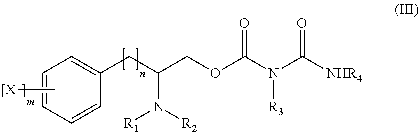

wherein: X is independently halo, alkyl, alkoxy or nitro; m is 0, 1, 2, 3 or 4; n is 1 or 2; R₁ and R₂ are independently H— or alkyl; R₃ is H—, alkyl or aralkyl; and R₄ is H— or aryl. In some embodiments, at least one of R₁, R₂, R₃ and R₄ is not H—.

In an embodiment, X is halo, C1-C4 alkyl, C1-C4 alkoxy or nitro; R₁ and R₂ are independently H— or C1-C4 alkyl; R₃ is H—, C1-C4 alkyl or C6-C10 aryl-C1-C4 alkyl; and R₄ is H— or C6-C10 aryl.

X is one or more substituents attached to the phenyl group of Formula (III) where m is a number of X substitutions. Thus, the phenyl group has up to 4 substituents selected from X. In an embodiment, X is independently selected from halo, methyl, tert-butyl, ethoxy and nitro.

n represents a number of carbon atom(s) of the alkylene linker; in other words, n=1 and n=2 represent —CH₂— and —CH₂CH₂—, respectively.

In an embodiment, R₁ and R₂ are independently H—, methyl or isopropyl.

In an embodiment, R₃ is methyl, ethyl, or benzyl. In a particular embodiment, R3 is methyl.

In an embodiment, R₄ is H— or phenyl. In a particular embodiment, R4 is H—.

In particular embodiments, compounds of Formula (III) include, but not limited to, the following compounds: 2-(isopropylamino)-3-phenylpropyl (aminocarbonyl)carbamate; 2-(dimethylamino)-3-phenylpropyl (aminocarbonyl)carbamate; 2-amino-3-(2-chlorophenyl)propyl (aminocarbonyl)carbamate; 2-amino-3-(2,4-dichlorophenyl)propyl (aminocarbonyl)carbamate; 2-amino-3-(3,4-dichlorophenyl)propyl (aminocarbonyl)carbamate; 2-amino-3-phenylpropyl (aminocarbonyl)methylcarbamate; 2-amino-3-(4-chlorophenyl)propyl (aminocarbonyl)carbamate; 2-amino-3-(3-chlorophenyl)propyl (aminocarbonyl)carbamate; 2-amino-3-(4-nitrophenyl)propyl (aminocarbonyl)carbamate; 2-amino-3-(4-tert-butylphenyl)propyl (aminocarbonyl)carbamate; 2-amino-3-(2-fluorophenyl)propyl (aminocarbonyl)carbamate; 2-(methylamino)-3-phenylpropyl (aminocarbonyl)carbamate; 2-(dimethylamino)-3-phenylpropyl (aminocarbonyl)methylcarbamate; 2-amino-3-phenylpropyl (aminocarbonyl)benzylcarbamate; 2-amino-3-phenylpropyl (aminocarbonyl)ethylcarbamate; 2-amino-3-(2-chlorophenyl)propyl (aminocarbonyl)methylcarbamate; 2-amino-3-(4-fluorophenyl)propyl (aminocarbonyl)methylcarbamate; 2-amino-3-(4-chlorophenyl)propyl (aminocarbonyl)methylcarbamate; 2-amino-3-(2,4-dichlorophenyl)propyl (aminocarbonyl)methylcarbamate; 2-amino-3-(3,4-dichlorophenyl)propyl (aminocarbonyl)methylcarbamate; 2-amino-3-phenylpropyl (aminocarbonyl)methylcarbamate; 2-amino-3-(4-nitrophenyl)propyl (aminocarbonyl)methylcarbamate; 2-amino-3-(4-methylphenyl)propyl (aminocarbonyl)methylcarbamate; 2-amino-3-(4-ethoxyphenyl)propyl (aminocarbonyl)methylcarbamate; 2-amino-4-phenylbutyl (aminocarbonyl)methylcarbamate; and 2-amino-3-phenylpropyl (anilinocarbonyl)carbamate.

Compounds of Formula (III) include all permissible isomers such as racemates, enantiomers, diastereomers and isotopic variants. In some embodiments, a compound of Formula (III) is a stereoisomer. In a particular embodiment, the stereoisomer is substantially enantiopure, for example consisting essentially of the R enantiomer of the compound. Examples of enantiomeric compounds include, but are not limited to, the following compounds: (2R)-2-(isopropylamino)-3-phenylpropyl (aminocarbonyl)carbamate; (2R)-2-(dimethylamino)-3-phenylpropyl (aminocarbonyl)carbamate; (2R)-2-amino-3-(2-chlorophenyl)propyl (aminocarbonyl)carbamate; (2R)-2-amino-3-(2,4-dichlorophenyl)propyl (aminocarbonyl)carbamate; (2R)-2-amino-3-(3,4-dichlorophenyl)propyl (aminocarbonyl)carbamate; (2R)-2-amino-3-phenylpropyl (aminocarbonyl)methylcarbamate; (2R)-2-(methylamino)-3-phenylpropyl (aminocarbonyl)carbamate; (2R)-2-(dimethylamino)-3-phenylpropyl (aminocarbonyl)methylcarbamate; (2R)-2-amino-3-phenylpropyl (aminocarbonyl)benzylcarbamate; (2R)-2-amino-3-phenylpropyl (aminocarbonyl)ethylcarbamate; (2R)-2-amino-3-(2-chlorophenyl)propyl (aminocarbonyl)methylcarbamate; (2R)-2-amino-3-(4-chlorophenyl)propyl (aminocarbonyl)methylcarbamate; (2R)-2-amino-3-(2,4-dichlorophenyl)propyl (aminocarbonyl)methylcarbamate; (2R)-2-amino-3-(3,4-dichlorophenyl)propyl (aminocarbonyl)methylcarbamate; (2S)-2-amino-3-phenylpropyl (aminocarbonyl)methylcarbamate; (2R)-2-amino-3-(4-nitrophenyl)propyl (aminocarbonyl)methylcarbamate; (2R)-2-amino-3-(4-methylphenyl)propyl (aminocarbonyl)methylcarbamate; (2R)-2-amino-3-(4-ethoxyphenyl)propyl (aminocarbonyl)methylcarbamate; (2R)-2-amino-4-phenylbutyl (aminocarbonyl)methylcarbamate; and (2R)-2-amino-3-phenylpropyl (anilinocarbonyl)carbamate.

In another embodiment, there is provided a compound of Formula (IV) or a pharmaceutically acceptable salt thereof:

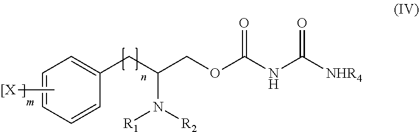

wherein X, m, n, R₁, R₂, and R₄ are as defined above. In some embodiments, at least one of R₁, R₂ and R₄ is not H—.

In an embodiment, X is halo, C1-C4 alkyl, C1-C4 alkoxy or nitro; R₁ and R₂ are independently H— or C1-C4 alkyl; and R₄ is H— or C6-C10 aryl.

In an embodiment, X is independently halo, methyl, tert-butyl, ethoxy or nitro. In a particular embodiment, X is chloro, fluoro, methyl, tert-butyl, ethoxy or nitro.

In an embodiment, R₁ and R₂ are independently H—, methyl or isopropyl. In particular embodiments, R₁ is methyl and R₂ is H—; R₁ is methyl and R₂ is methyl; or R₁ is isopropyl and R₂ is H—.

In an embodiment, R₄ is H— or phenyl.

Examples of Formula (IV) includes, but are not limited to, the following compounds: 2-(isopropylamino)-3-phenylpropyl (aminocarbonyl)carbamate; 2-(dimethylamino)-3-phenylpropyl (aminocarbonyl)carbamate; 2-amino-3-(2-chlorophenyl)propyl (aminocarbonyl)carbamate; 2-amino-3-(2,4-dichlorophenyl)propyl (aminocarbonyl)carbamate; 2-amino-3-(3,4-dichlorophenyl)propyl (aminocarbonyl)carbamate; 2-amino-3-(4-chlorophenyl)propyl (aminocarbonyl)carbamate; 2-amino-3-(3-chlorophenyl)propyl (aminocarbonyl)carbamate; 2-amino-3-(4-nitrophenyl)propyl (aminocarbonyl)carbamate; 2-amino-3-(4-tert-butylphenyl)propyl (aminocarbonyl)carbamate; 2-amino-3-(2-fluorophenyl)propyl (aminocarbonyl)carbamate; 2-(methylamino)-3-phenylpropyl (aminocarbonyl)carbamate; and 2-amino-3-phenylpropyl (anilinocarbonyl)carbamate.

Compounds of Formula (IV) include all permissible isomers. In some embodiment, a compound of Formula (IV) is an enantiomer. In a particular embodiment, the stereoisomer is substantially enantiopure, for example consisting essentially of the R enantiomer of the compound. Examples of enantiomers include, but are not limited to, the following compounds: (2R)-2-(isopropylamino)-3-phenylpropyl (aminocarbonyl)carbamate; (2R)-2-(dimethylamino)-3-phenylpropyl (aminocarbonyl)carbamate; (2R)-2-amino-3-(2-chlorophenyl)propyl (aminocarbonyl)carbamate; (2R)-2-amino-3-(2,4-dichlorophenyl)propyl (aminocarbonyl)carbamate; (2R)-2-amino-3-(3,4-dichlorophenyl)propyl (aminocarbonyl)carbamate; (2R)-2-(methylamino)-3-phenylpropyl (aminocarbonyl)carbamate; and (2R)-2-amino-3-phenylpropyl (anilinocarbonyl)carbamate.

In yet another embodiment, there is provided a compound of Formula (V) or a pharmaceutically acceptable salt thereof:

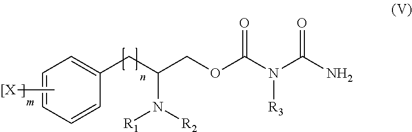

wherein X, m, n, R₁, R₂, and R₃ are as defined above. In some embodiments, at least one of R₁, R₂ and R₃ is not H—.

In an embodiment, X is halo, C1-C4 alkyl, C1-C4 alkoxy or nitro; R₁ and R₂ are independently H— or C1-C4 alkyl; and R₃ is H—, C1-C4 alkyl or C6-C10 aryl-C1-C4 alkyl.

In an embodiment, X is independently halo, methyl, tert-butyl, ethoxy or nitro. In a particular embodiment, X is chloro, fluoro, methyl, tert-butyl, ethoxy or nitro.

In an embodiment, R₁ and R₂ are independently H—, methyl or isopropyl.

In an embodiment, R₃ is H—, C1-C4 alkyl or C6-C10 aryl-C1-C4 alkyl, particularly methyl, ethyl, or benzyl. In a particular embodiment, R3 is methyl.

Examples of Formula (V) includes, but are not limited to, the following compounds: 2-amino-3-phenylpropyl (aminocarbonyl)methylcarbamate; 2-(dimethylamino)-3-phenylpropyl (aminocarbonyl)methylcarbamate; 2-amino-3-phenylpropyl (aminocarbonyl)benzylcarbamate; 2-amino-3-phenylpropyl (aminocarbonyl)ethylcarbamate; 2-amino-3-(2-chlorophenyl)propyl (aminocarbonyl)methylcarbamate; 2-amino-3-(4-fluorophenyl)propyl (aminocarbonyl)methylcarbamate; 2-amino-3-(4-chlorophenyl)propyl (aminocarbonyl)methylcarbamate; 2-amino-3-(2,4-dichlorophenyl)propyl (aminocarbonyl)methylcarbamate; 2-amino-3-(3,4-dichlorophenyl)propyl (aminocarbonyl)methylcarbamate; 2-amino-3-(4-nitrophenyl)propyl (aminocarbonyl)methylcarbamate; 2-amino-3-(4-methylphenyl)propyl (aminocarbonyl)methylcarbamate; 2-amino-3-(4-ethoxyphenyl)propyl (aminocarbonyl)methylcarbamate; and 2-amino-4-phenylbutyl (aminocarbonyl)methylcarbamate.

Compounds of Formula (V) include all permissible isomers. In some embodiment, a compound of Formula (V) is an enantiomer. In a particular embodiment, the stereoisomer is substantially enantiopure, for example consisting essentially of the R enantiomer of the compound. Examples of enantiomers include, but are not limited to, the following compounds: (2R)-2-amino-3-phenylpropyl (aminocarbonyl)methylcarbamate; (2R)-2-(dimethylamino)-3-phenylpropyl (aminocarbonyl)methylcarbamate; (2R)-2-amino-3-phenylpropyl (aminocarbonyl)benzylcarbamate; (2R)-2-amino-3-phenylpropyl (aminocarbonyl)ethylcarbamate; (2R)-2-amino-3-(2-chlorophenyl)propyl (aminocarbonyl)methylcarbamate; (2R)-2-amino-3-(4-chlorophenyl)propyl (aminocarbonyl)methylcarbamate; (2R)-2-amino-3-(2,4-dichlorophenyl)propyl (aminocarbonyl)methylcarbamate; (2R)-2-amino-3-(3,4-dichlorophenyl)propyl (aminocarbonyl)methylcarbamate; (2S)-2-amino-3-phenylpropyl (aminocarbonyl)methylcarbamate; (2R)-2-amino-3-(4-nitrophenyl)propyl (aminocarbonyl)methylcarbamate; (2R)-2-amino-3-(4-methylphenyl)propyl (aminocarbonyl)methylcarbamate; (2R)-2-amino-3-(4-ethoxyphenyl)propyl (aminocarbonyl)methylcarbamate; and (2R)-2-amino-4-phenylbutyl (aminocarbonyl)methylcarbamate.

In yet another embodiment, there is provided 2-amino-3-phenylpropyl (aminocarbonyl)carbamate or a pharmaceutically acceptable salt thereof. In some embodiments, the compound is an enantiomer of 2-amino-3-phenylpropyl (aminocarbonyl)carbamate, such as, (2R)-2-amino-3-phenylpropyl (aminocarbonyl)carbamate.

Other embodiments of the invention include the use, for the preparation of a medicament for ameliorating biomarkers associated with cardiovascular risk, of one of the compounds or enantiomers or enantiomeric mixtures described above or a pharmaceutically acceptable salt or ester thereof.

All of the U.S. patents that have been mentioned above in connection with compounds used in the present invention are incorporated herein by reference.

### III. Functional Aspects and Other Characteristics of APC and Compositions Thereof

As will be appreciated, discussion herein of functional aspects and other characteristics of APC can also apply to compositions comprising APC of the presently disclosed invention as well as to compounds related to APC known in the art and described in detail herein.

In some aspects, an APC described herein may improve the levels of biomarkers associated with cardiovascular risk. In some embodiments, biomarkers associated with cardiovascular risk include blood pressure, blood dipping, triglycerides, cholesterol, glucose and insulin levels, C-reactive protein (CRP), body weight, and body mass index (BMI). (O'Donnell et al., Cardiovascular Risk Factors. Insights From Framingham Heart Study, Rev Esp Cardiol. 2008; 61(3):299-310).

### A. Body Mass Index (BMI) and Body Weight

In some aspects, an APC described herein may reduce body mass index (BMI). BMI is defined as a person's weight in kg divided by height in meters. There are four BMI classifications (www.cdc.gov/healthyweight/assessing/bmi/adult_bmi/index.html). A patient with a BMI of below 18.5 is classified as underweight. A patient with a BMI of 18.5-24.9 is classified as normal. A patient with a BMI of 25-29.9 is classified as overweight. A patient with a BMI of over 30 is considered obese (Prospective Studies Collaboration 2009; Clinical Guidelines on the Identification, Evaluation, and Treatment of Overweight and Obesity in Adults—The Evidence Report. National Institutes of Health; Obesity: preventing and managing the global epidemic. Report of a WHO consultation). High BMI measurements are associated with a higher risk for development of cardiovascular diseases and mortality due to cardiovascular diseases (Khan et al., Association of Body Mass Index With Lifetime Risk of Cardiovascular Disease and Compression of Morbidity. JAMA Cardiol. 2018 Apr. 1; 3(4):280-287, Katzmarzyk et al., Body Mass Index and Risk of Cardiovascular Disease, Cancer and All-cause Mortality, Can J Public Health. 2012 March; 103(2): 147-151). In some embodiments, administration of APC reduces BMI by 5% or more. In some embodiments, administration of APC reduces BMI by about 5%. In some embodiments, administration of APC reduces BMI by less than 5% but does not increase BMI.

In some aspects, an APC described herein may reduce body weight. In some embodiments, administration of APC reduces body weight by 5% or more. In some embodiments, administration of APC reduces body weight by about 5%. In some embodiments, administration of APC reduces body weight by less than 5%, but does not increase body weight.

In some embodiments, the patient has been receiving (R)-2-amino-3-phenylpropylcarbamate (APC) for any number of weeks and has over that number of weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 54 weeks and has over that 5 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 52 weeks and has over that 52 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 50 weeks and has over that 50 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 48 weeks and has over that 48 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 44 weeks and has over that 44 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 42 weeks and has over that 42 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 41 weeks and has over that 41 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 40 weeks and has over that 40 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 39 weeks and has over that 39 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 38 weeks and has over that 38 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 37 weeks and has over that 37 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 36 weeks and has over that 36 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 30 weeks and has over that 30 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 26 weeks and has over that 26 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 24 weeks and has over that 24 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 20 weeks and has over that 20 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 16 weeks and has over that 16 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 12 weeks and has over that 12 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 8 weeks and has over that 8 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 6 weeks and has over that 6 weeks experienced at least about 5% loss in BMI as compared to before receiving (R)-2-amino-3-phenylpropylcarbamate (APC). In some embodiments, the patient has been receiving (R)-2-amino-3-phenylpropylcarbamate (APC) for at least 4 weeks and has over that 4 weeks experienced at least about 5% loss in BMI as compared to before receiving (R)-2-amino-3-phenylpropylcarbamate (APC).

In some embodiments, the patient has been receiving (R)-2-amino-3-phenylpropylcarbamate (APC) for any number of weeks and has over that number of weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 54 weeks and has over that 54 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 52 weeks and has over that 52 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 50 weeks and has over that 50 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 48 weeks and has over that 48 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 44 weeks and has over that 44 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 42 weeks and has over that 42 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 40 weeks and has over that 41 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 40 weeks and has over that 40 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 39 weeks and has over that 39 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 38 weeks and has over that 38 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 37 weeks and has over that 37 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 36 weeks and has over that 36 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 30 weeks and has over that 30 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 26 weeks and has over that 26 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 24 weeks and has over that 24 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 20 weeks and has over that 20 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 16 weeks and has over that 16 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 12 weeks and has over that 12 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 8 weeks and has over that 8 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 6 weeks and has over that 6 weeks experienced at least about 5% loss in body weight as compared to before receiving (R)-2-amino-3-phenylpropylcarbamate (APC). In some embodiments, the patient has been receiving (R)-2-amino-3-phenylpropylcarbamate (APC) for at least 4 weeks and has over that 4 weeks experienced at least about 5% loss in body weight as compared to before receiving (R)-2-amino-3-phenylpropylcarbamate (APC).

In some embodiments, an APC ameliorates at least one biomarker of cardiovascular risk in a patient that has a BMI of 25 kg/m² or greater. In some embodiments, an APC ameliorates at least one biomarker of cardiovascular risk in a patient that has a BMI in the range from 25 kg/m² to less than 30 kg/m². In some embodiments, an APC ameliorates at least one biomarker of cardiovascular risk in a patient that has a BMI in the range from 30 kg/m² or greater.

### B. Blood Pressure

High blood pressure contributes to cardiovascular disease. Patients with abnormally high blood pressure are given the diagnosis hypertension, and generally prescribed antihypertensives to manage blood pressure levels. Over time, high blood pressure has been shown to increase the risk of cardiovascular diseases, in particular stroke and coronary heart diseases (Wu et al., High Blood Pressure and All-Cause and Cardiovascular Disease Mortalities in Community-Dwelling Older Adults, Medicine (Baltimore). 2015 November; 94(47): e2160). According to the American College of Cardiology, blood pressure is classified into one of five categories (American College of Cardiology, New ACC/AHA High Blood Pressure Guidelines Lower Definition of Hypertension, published online Nov. 13, 2017 at www.acc.org/latest-in-cardiology/articles/2017/11/08/11/47/mon-5 pm-bp-guideline-aha-2017). Normal blood pressure is found in patients who have a systolic blood pressure of 120 mm Hg and a diastolic blood pressure of 80 mm Hg. Elevated blood pressure is found in patients who have a systolic blood pressure between 120-129 mm Hg and diastolic blood pressure less than 80 mm Hg. Stage 1 hypertension is found in patients who have a systolic blood pressure between 130-139 mmHg or a diastolic blood pressure of between 80-89 mm Hg. Stage 2 hypertension is found in patients who have a systolic blood pressure that is at least 140 mm Hg or a diastolic blood pressure that is at least 90 mm Hg. Hypertensive crisis is found in patients who have systolic blood pressure over 180 mm Hg and/or diastolic blood pressure over 120 mm Hg. In some aspects, an APC described herein reduces blood pressure. In some embodiments, an APC may reduce blood pressure level in patients with a BMI above the normal level. In some embodiments, an APC may reduce blood pressure level in patients who lose greater than or equal to 5% of their weight during treatment with APC. In some embodiments, an APC may reduce blood pressure level in patients who lose less than 5% of their weight during treatment with APC. In some embodiments, an APC reduces blood pressure level by reducing systolic blood pressure. In some embodiments, an APC reduces blood pressure level by reducing diastolic blood pressure. In some embodiments, an APC reduces blood pressure level by reducing both systolic blood pressure and diastolic blood pressure. In some embodiments, an APC causes no change in blood pressure level.

In some embodiments, a reduction in blood pressure level may be 30 mm Hg or greater. In some embodiments, a reduction in blood pressure level may be 20 mm Hg or greater. In some embodiments, a reduction in blood pressure level may be 10 mm Hg or greater. In some embodiments, a reduction in blood pressure level may be 2 mm Hg or greater. In some embodiments, a reduction in blood pressure level may be 1 mm Hg or greater. In some embodiments, a reduction blood pressure level is less than 1 mm Hg.

In some embodiments, a reduction in systolic blood pressure may be 30 mm Hg or greater. In some embodiments, a reduction in systolic blood pressure may be 20 mm Hg or greater. In some embodiments, a reduction in systolic blood pressure may be 10 mm Hg or greater. In some embodiments, a reduction in systolic blood pressure may be 2 mm Hg or greater. In some embodiments, a reduction in systolic blood pressure may be 1 mm Hg or greater. In some embodiments, a reduction in systolic blood pressure is less than 1 mm Hg.

In some embodiments, a reduction in diastolic blood pressure may be 30 mm Hg or greater. In some embodiments, a reduction in diastolic blood pressure may be 20 mm Hg or greater. In some embodiments, a reduction in diastolic blood pressure may be 10 mm Hg or greater. In some embodiments, a reduction in diastolic blood pressure may be 2 mm Hg or greater. In some embodiments, a reduction in diastolic blood pressure may be 1 mm Hg or greater. In some embodiments, a reduction in diastolic blood pressure is less than 1 mm Hg.

In some embodiments, a reduction in blood pressure may lower the patient's blood pressure classification. In some embodiments, the patient had high blood pressure prior to treatment with APC and after treatment with APC the patient has normal blood pressure. In some embodiments, the patient had high blood pressure prior to treatment with APC and after treatment with APC the patient still has high blood pressure, but the blood pressure is reduced. In some embodiments, the patient had normal blood pressure prior to treatment with APC and blood pressure in the patient remains normal after treatment with APC. In some embodiments, the patient has no increase in blood pressure after treatment of APC. Example 1 provides data regarding the ability of APC to reduce blood pressure in patients treated with APC.

In some embodiments, an APC reduces blood pressure in a patient that has a BMI of 25 kg/m² or greater. In some embodiments, an APC reduces blood pressure in a patient that has a BMI in the range from 25 kg/m² to less than 30 kg/m². In some embodiments, an APC reduces blood pressure in a patient has a BMI in the range from 30 kg/m² or greater.

In some embodiments, the patient has been receiving (R)-2-amino-3-phenylpropylcarbamate (APC) for any number of weeks and has over that number of weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 54 weeks and has over that 54 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 52 weeks and has over that 52 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 50 weeks and has over that 50 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 48 weeks and has over that 48 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 44 weeks and has over that 44 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 42 weeks and has over that 42 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 40 weeks and has over that 41 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 40 weeks and has over that 40 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 39 weeks and has over that 39 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 38 weeks and has over that 38 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 37 weeks and has over that 37 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 36 weeks and has over that 36 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 30 weeks and has over that 30 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 26 weeks and has over that 26 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 24 weeks and has over that 24 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 20 weeks and has over that 20 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 16 weeks and has over that 16 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 12 weeks and has over that 12 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 8 weeks and has over that 8 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 6 weeks and has over that 6 weeks experienced a reduction in blood pressure as compared to before receiving (R)-2-amino-3-phenylpropylcarbamate (APC). In some embodiments, the patient has been receiving (R)-2-amino-3-phenylpropylcarbamate (APC) for at least 4 weeks and has over that 4 weeks experienced a reduction in blood pressure as compared to before receiving (R)-2-amino-3-phenylpropylcarbamate (APC).

i. Blood Dipping

Blood dipping is the ratio of mean nighttime blood pressure (sleep) to mean daytime blood pressure (awake). People with a blood dipping ratio of greater than or equal to 0.90 or defined as <10% decrease in mean arterial pressure (MAP) during sleep versus MAP while awake are considered to be classified as non-dipping and have increased risk of cardiovascular events (Taylor K S et al., Heterogeneity of Prognostic Studies of 24-hour Blood Pressure Variability: Systematic Review and Meta-Analysis. Plos One (2015) 10(5):e0126375; Viera et al., Nighttime blood pressure dipping in young adults and coronary artery calcium 10-15 years later: the coronary artery risk development in young adults study, Hypertension. 2012 June; 59(6):1157-63). In some embodiments, after administration of APC, the patient does not display an increased propensity toward a non-dipping classification over the course of treatment. In some embodiments, the patient is classified as non-dipping prior to treatment with APC, and after the patient is administered an APC, the patient is no longer classified as non-dipping. In some embodiments, the patient is not classified as non-dipping, and after the patient is administered an APC, the patient will still not be classified as non-dipping. Example 2 provides data regarding the effect of APC on blood dipping classifications of patients treated with APC.

In some embodiments, an APC reduces blood dipping ratio in a patient that has a BMI of 25 kg/m² or greater. In some embodiments, an APC reduces blood dipping ratio in a patient that has a BMI in the range from 25 kg/m² to less than 30 kg/m². In some embodiments, an APC reduces blood dipping ratio in a patient has a BMI in the range from 30 kg/m² or greater.

In some embodiments, the patient has been receiving (R)-2-amino-3-phenylpropylcarbamate (APC) for any number of weeks and has over that number of weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 54 weeks and has over that 54 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 52 weeks and has over that 52 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 50 weeks and has over that 50 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 48 weeks and has over that 48 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 44 weeks and has over that 44 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 42 weeks and has over that 42 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 40 weeks and has over that 41 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 40 weeks and has over that 40 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 39 weeks and has over that 39 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 38 weeks and has over that 38 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 37 weeks and has over that 37 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 36 weeks and has over that 36 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 30 weeks and has over that 30 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 26 weeks and has over that 26 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 24 weeks and has over that 24 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 20 weeks and has over that 20 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 16 weeks and has over that 16 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 12 weeks and has over that 12 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 8 weeks and has over that 8 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 6 weeks and has over that 6 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for at least 4 weeks and has over that 4 weeks experienced a reduction in blood dipping ratio as compared to before receiving (R)-2-amino-3-phenylpropylcarbamate (APC). In some embodiments, the patient has been receiving (R)-2-amino-3-phenylpropylcarbamate (APC) for any number of weeks and has over that number of weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 54 weeks and has over that 54 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 52 weeks and has over that 52 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 50 weeks and has over that 50 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 48 weeks and has over that 48 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 44 weeks and has over that 44 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 42 weeks and has over that 42 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 40 weeks and has over that 41 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 40 weeks and has over that 40 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 39 weeks and has over that 39 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 38 weeks and has over that 38 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 37 weeks and has over that 37 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 36 weeks and has over that 36 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 30 weeks and has over that 30 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 26 weeks and has over that 26 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 24 weeks and has over that 24 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 20 weeks and has over that 20 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 16 weeks and has over that 16 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 12 weeks and has over that 12 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 8 weeks and has over that 8 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 6 weeks and has over that 6 weeks experienced a reduction in MAP during sleep versus MAP while awake that is greater than or equal to 10% as compared to before receiving (R)-2-amino-3-phenylpropylcarbamate (APC). In some embodiments, the patient has been receiving (R)-2-amino-3-phenylpropylcarbamate (APC) for at least 4 weeks and has over that 4 weeks experienced a reduction in MAP during sleep versus MAP while awake that is greater than or equal to 10% as compared to before receiving (R)-2-amino-3-phenylpropylcarbamate (APC).

### C. Triglycerides

In some aspects, an APC described herein may reduce triglycerides. High levels of triglycerides are associated with cardiovascular disease (Marston et al, Association Between Triglyceride Lowering and Reduction of Cardiovascular Risk Across Multiple Lipid-Lowering Therapeutic Classes, Circulation. 2019; 140:1308-1317). According to the National Library of Medicine, there are four classifications for triglyceride levels in a patient. Triglyceride levels less than 150 mg/dL are considered normal in a patient. Triglyceride levels of 150-199 mg/dL are considered borderline high in a patient. Triglyceride levels of 200-499 mg/dL are considered high in a patient. Triglyceride levels of 500 mg/dL or above are considered very high. Any level above normal is considered to be a higher risk for cardiovascular disease (US National Library of Medicine, Medline Plus: Triglycerides at medlineplus.gov/triglycerides.html).

In some embodiments, an APC described herein may reduce triglyceride levels in a patient. In some embodiments, an APC may reduce triglyceride levels in patients with a BMI above the normal level. In some embodiments, an APC may reduce triglyceride levels in patients who lose greater than 5% of their weight during treatment with APC. In some embodiments, an APC may reduce triglyceride levels in patients who lose less than 5% of their weight during treatment with APC. In some embodiments, the patient had high triglyceride levels prior to treatment with APC, and after treatment with APC, the patient has normal triglyceride levels. In some embodiments, an APC described herein lowers triglyceride levels in a patient to cause the patient to fall into a lower classification. In some embodiments, an APC described herein lowers triglyceride levels in a patient but does not cause the patient to fall into a lower classification. In some embodiments, the patient had a normal triglyceride level prior to treatment of APC and the level remains normal after treatment with APC. In some embodiments, a patient experiences no increase in triglyceride level after treatment with APC. Example 1 provides data regarding the ability of APC to reduce triglyceride levels in patients treated with APC.

In some embodiments, an APC reduces triglyceride levels in a patient that has a BMI of 25 kg/m² or greater. In some embodiments, an APC reduces triglyceride levels in a patient that has a BMI in the range from 25 kg/m² to less than 30 kg/m². In some embodiments, an APC reduces triglyceride levels in a patient has a BMI in the range from 30 kg/m² or greater.

In some embodiments, the patient has been receiving (R)-2-amino-3-phenylpropylcarbamate (APC) for any number of weeks and has over that number of weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 54 weeks and has over that 54 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 52 weeks and has over that 52 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 50 weeks and has over that 50 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 48 weeks and has over that 48 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 44 weeks and has over that 44 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 42 weeks and has over that 42 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 40 weeks and has over that 41 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 40 weeks and has over that 40 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 39 weeks and has over that 39 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 38 weeks and has over that 38 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 37 weeks and has over that 37 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 36 weeks and has over that 36 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 30 weeks and has over that 30 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 26 weeks and has over that 26 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 24 weeks and has over that 24 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 20 weeks and has over that 20 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 16 weeks and has over that 16 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 12 weeks and has over that 12 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 8 weeks and has over that 8 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 6 weeks and has over that 6 weeks experienced a reduction in triglyceride levels as compared to before receiving (R)-2-amino-3-phenylpropylcarbamate (APC). In some embodiments, the patient has been receiving (R)-2-amino-3-phenylpropylcarbamate (APC) for at least 4 weeks and has over that 4 weeks experienced a reduction in triglyceride levels as compared to before receiving (R)-2-amino-3-phenylpropylcarbamate (APC).

### D. Cholesterol

In some aspects, an APC described herein may improve the cholesterol levels of a patient. High levels of cholesterol have been associated with an increased risk of cardiovascular disease, particularly levels of low density lipoprotein (LDL) cholesterol (Benjamin et al., Heart Disease and Stroke Statistics—2019 Update: A Report From the American Heart Association, Circulation. 2019; 139:e56-e528). Studies suggest that an ideal blood cholesterol level is 150 mg/dL total cholesterol, with an LDL cholesterol level of at or below 100 mg/dL (Grundy S M, Stone N J, Bailey A L, et al. 2018 AHA/ACC/AACVPR/AAPA/ABC/ACPM/ADA/AGS/

APhA/ASPC/NLA/PCNA guideline on the management of blood cholesterol: a report of the American College of Cardiology/American Heart Association Task Force on Clinical Practice Guidelines [published online Nov. 10, 2018]. Circulation.). Studies show that adults at this level have lower rates of cardiovascular disease (American Heart Association, Cholesterol Management Guide, published online 2018, www.heart.org/-/media/files/health-topics/cholesterol/chlstrmngmntgd_181110.pdf). Another way to measure cholesterol is using cholesterol ratios. Cholesterol ratio is measured by dividing total cholesterol number by high density lipoprotein (HDL) level. Higher ratios mean there is a higher risk of heart disease.

In some embodiments, an APC reduces cholesterol. In some embodiments, an APC reduces total cholesterol. In some embodiments, an APC reduces total cholesterol in patients with a BMI above the normal level. In some embodiments, an APC reduces total cholesterol in patients who lose greater than 5% of their weight during treatment with APC. In some embodiments, an APC reduces total cholesterol in patients who lose less than 5% of their weight during treatment with APC. In some embodiments, a patient experiences no increase in total cholesterol after treatment with APC. In some embodiments, an APC reduces low density lipoprotein (LDL) cholesterol. In some embodiments, an APC reduces LDL cholesterol in patients with a BMI above the normal level. In some embodiments, an APC reduces LDL cholesterol in patients who lose greater than 5% of their weight during treatment with APC. In some embodiments, an APC may reduce LDL cholesterol in patients who lose less than 5% of their weight during treatment with APC. In some embodiments, a patient experiences no increase in LDL cholesterol after treatment with APC. In some embodiments, an APC reduces cholesterol ratio. In some embodiments, an APC reduces cholesterol ratio in patients with a BMI above the normal level. In some embodiments, an APC reduces cholesterol ratio in patients who lose greater than 5% of their weight during treatment with APC. In some embodiments, an APC reduces cholesterol ratio in patients who lose less than 5% of their weight during treatment with APC. In some embodiments, a patient experiences no increase in cholesterol ratio after treatment with APC.

In some embodiments, an APC reduces total cholesterol in a patient that has a BMI of 25 kg/m² or greater. In some embodiments, an APC reduces total cholesterol in a patient that has a BMI in the range from 25 kg/m² to less than 30 kg/m². In some embodiments, an APC reduces total cholesterol in a patient has a BMI in the range from 30 kg/m² or greater. In some embodiments, an APC reduces LDL cholesterol in a patient that has a BMI of 25 kg/m² or greater. In some embodiments, an APC reduces LDL cholesterol in a patient that has a BMI in the range from 25 kg/m² to less than 30 kg/m². In some embodiments, an APC reduces LDL cholesterol in a patient has a BMI in the range from 30 kg/m² or greater. In some embodiments, an APC reduces cholesterol ratio in a patient that has a BMI of 25 kg/m² or greater. In some embodiments, an APC reduces cholesterol ratio in a patient that has a BMI in the range from 25 kg/m² to less than 30 kg/m². In some embodiments, an APC reduces cholesterol ratio in a patient has a BMI in the range from 30 kg/m² or greater.

In some embodiments, the patient has been receiving (R)-2-amino-3-phenylpropylcarbamate (APC) for any number of weeks and has over that number of weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 54 weeks and has over that 54 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 52 weeks and has over that 52 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 50 weeks and has over that 50 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 48 weeks and has over that 48 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 44 weeks and has over that 44 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 42 weeks and has over that 42 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 40 weeks and has over that 41 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 40 weeks and has over that 40 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 39 weeks and has over that 39 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 38 weeks and has over that 38 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 37 weeks and has over that 37 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 36 weeks and has over that 36 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 30 weeks and has over that 30 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 26 weeks and has over that 26 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 24 weeks and has over that 24 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 20 weeks and has over that 20 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 16 weeks and has over that 16 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 12 weeks and has over that 12 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 8 weeks and has over that 8 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 6 weeks and has over that 6 weeks experienced a reduction in cholesterol levels as compared to before receiving (R)-2-amino-3-phenylpropylcarbamate (APC). In some embodiments, the patient has been receiving (R)-2-amino-3-phenylpropylcarbamate (APC) for at least 4 weeks and has over that 4 weeks experienced a reduction in cholesterol levels as compared to before receiving (R)-2-amino-3-phenylpropylcarbamate (APC).

### E. Glucose

In some aspects, an APC described herein may reduce glucose levels in a patient. High blood glucose levels are associated with increased risk of developing cardiovascular disease and many of these patients are also diagnosed as diabetics (Lawes et al., Blood glucose and risk of cardiovascular disease in the Asia Pacific region, Diabetes Care. 2004 December; 27(12):2836-42). There are multiple different tests that may be used to measure glucose levels, these include, but are not limited to: fasting blood glucose test, oral glucose tolerance test, insulin test, two-hour post-prandial test, random blood glucose test, homeostatic model assessment of insulin resistance (HOMA-IR), and hemoglobin A1C (hbA1C) test. Glucose tests may be performed using urine or blood. A patient with a result out of the normal range for any glucose test may be at higher risk for cardiovascular disease. Fasting blood glucose tests are taken after an overnight fast (no food or drink) and a glucose level of less than 100 mg/dL (5.6 mmol/L) is considered normal, a glucose level from 100 mg/dL to 125 mg/dL (5.6 mmol/L to 6.9 mmol/L) is considered pre-diabetic, and a glucose level of 126 mg/dL (7.0 mmol/L) or higher is considered diabetic (Mayo Foundation for Medical Education and Research (MFMER), Diabetes, at www.mayoclinic.org/diseases-conditions/diabetes/diagnosis-treatment/drc-20371451). The normal range for a fasting insulin test is <25 mIU/L or <174 pmol/L. Random blood glucose tests may be given at any time after eating and a normal range is below 200 mg/dL or 11.1 mmol/L. HOMA-IR, is found by the formula ((insulin level (mg/dL)×glucose level (mg/dL))/(405)). Alternatively HOMA-IR may be found by the formula ((insulin level (mmol/L)×glucose level (mmol/L))/(22.5). HOMA-IR results greater than 2.5 are considered to be above normal. Hemoglobin A1C (hbA1C) have a normal level of less than 5.7%, the prediabetic level is between 5.7% and 6.4%, and the diabetic level is 6.5% or higher (US National Library of Medicine, Medline Plus: Hemoglobin A1C (HbA1c) Test at medlineplus.gov/lab-tests/hemoglobin-a1c-hba1c-test/).

In some embodiments, an APC described herein reduces glucose levels in a patient. In some embodiments, an APC may reduce glucose levels in patients with a BMI above the normal level. In some embodiments, an APC may reduce glucose levels in patients who lose greater than 5% of their weight during treatment with APC. In some embodiments, an APC may reduce glucose levels in patients who lose less than 5% of their weight during treatment with APC. In some embodiments, an APC reduces glucose levels in a patient with glucose levels above normal. In some embodiments, a patient had glucose levels above the normal level prior to treatment with an APC, and after treatment with the APC, the patient has normal glucose levels. In some embodiments, a patient experiences no increase in glucose levels after treatment with APC. Example 1 provides data regarding the ability of APC to reduce glucose levels in patients treated with APC.

In some embodiments, an APC reduces glucose levels in a patient that has a BMI of 25 kg/m² or greater. In some embodiments, an APC reduces glucose levels in a patient that has a BMI in the range from 25 kg/m² to less than 30 kg/m². In some embodiments, an APC reduces glucose levels in a patient has a BMI in the range from 30 kg/m² or greater.

In some embodiments, the patient has been receiving (R)-2-amino-3-phenylpropylcarbamate (APC) for any number of weeks and has over that number of weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 54 weeks and has over that 54 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 52 weeks and has over that 52 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 50 weeks and has over that 50 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 48 weeks and has over that 48 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 44 weeks and has over that 44 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 42 weeks and has over that 42 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 40 weeks and has over that 41 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 40 weeks and has over that 40 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 39 weeks and has over that 39 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 38 weeks and has over that 38 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 37 weeks and has over that 37 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 36 weeks and has over that 36 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 30 weeks and has over that 30 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 26 weeks and has over that 26 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 24 weeks and has over that 24 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 20 weeks and has over that 20 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 16 weeks and has over that 16 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 12 weeks and has over that 12 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 8 weeks and has over that 8 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 6 weeks and has over that 6 weeks experienced a reduction in glucose levels as compared to before receiving (R)-2-amino-3-phenylpropylcarbamate (APC). In some embodiments, the patient has been receiving (R)-2-amino-3-phenylpropylcarbamate (APC) for at least 4 weeks and has over that 4 weeks experienced a reduction in glucose levels as compared to before receiving (R)-2-amino-3-phenylpropylcarbamate (APC).

### F. C Reactive Protein

In some aspects, an APC described herein may reduce C Reactive Protein (CRP) in a patient. CRP is a protein made by the liver. CRP levels in the blood increase as a result of inflammation in the body and levels of CRP may indicate the presence of cardiovascular disease (Cozlea et al., The Impact of C Reactive Protein on Global Cardiovascular Risk on Patients with Coronary Artery Disease, Current Health Sciences Journal, 2013 October-December, 39(4): 225-231). A patient with a result out of the normal range for a CRP test may be at higher risk for cardiovascular disease. A CRP test may be performed with an erythrocyte sedimentation rate (ESR) or sedimentation rate test. High-sensitivity CRP (hs-CRP) tests may be used to determine cardiovascular risk. Generally, a low level of cardiovascular risk is associated with a hs-CRP test result of below 1.0 mg/L, a moderate level of cardiovascular risk is associated with a test result of between 1.0 mg/L and 3.0 mg/L, and a high level of cardiovascular risk is associated with a test result of greater than 3.0 mg/L (US National Library of Medicine, Medline Plus: C-reactive protein at medlineplus.gov/ency/article/003356.htm).

In some embodiments, an APC described herein reduces CRP levels in a patient. In some embodiments, an APC may reduce CRP levels in patients with a BMI above the normal level. In some embodiments, an APC may reduce CRP levels in patients who lose greater than 5% of their weight during treatment with APC. In some embodiments, an APC may reduce CRP levels in patients who lose less than 5% of their weight during treatment with APC. In some embodiments, an APC reduces CRP levels in a patient with CRP levels above low levels. In some embodiments, a patient had CRP levels above the low level prior to treatment with an APC, and after treatment with the APC, the patient has low CRP levels. In some embodiments, a patient experiences no increase in CRP levels after treatment with APC.

In some embodiments, an APC reduces CRP levels in a patient that has a BMI of 25 kg/m² or greater. In some embodiments, an APC reduces CRP levels in a patient that has a BMI in the range from 25 kg/m² to less than 30 kg/m². In some embodiments, an APC reduces CRP levels in a patient has a BMI in the range from 30 kg/m² or greater.

In some embodiments, the patient has been receiving (R)-2-amino-3-phenylpropylcarbamate (APC) for any number of weeks and has over that number of weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 54 weeks and has over that 54 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 52 weeks and has over that 52 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 50 weeks and has over that 50 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 48 weeks and has over that 48 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 44 weeks and has over that 44 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 42 weeks and has over that 42 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 40 weeks and has over that 41 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 40 weeks and has over that 40 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 39 weeks and has over that 39 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 38 weeks and has over that 38 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 37 weeks and has over that 37 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 36 weeks and has over that 36 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 30 weeks and has over that 30 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 26 weeks and has over that 26 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 24 weeks and has over that 24 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 20 weeks and has over that 20 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 16 weeks and has over that 16 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 12 weeks and has over that 12 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 8 weeks and has over that 8 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 6 weeks and has over that 6 weeks experienced a reduction in CRP levels as compared to before receiving (R)-2-amino-3-phenylpropylcarbamate (APC). In some embodiments, the patient has been receiving (R)-2-amino-3-phenylpropylcarbamate (APC) for at least 4 weeks and has over that 4 weeks experienced a reduction in CRP levels as compared to before receiving (R)-2-amino-3-phenylpropylcarbamate (APC).

The APC of the present disclosure may have any combination of the properties in the above sections or any other properties described herein.

### IV. Formulation of an APC

In some embodiments, the present disclosure provides methods for formulation of an APC.

Methods that may be used for formulation of an APC are described for example in U.S. Pat. Nos. 10,195,151 and 10,512,609; and U.S. Publication No. 2020/0085748 which are herein incorporated by reference in its entirety for all purposes and in particular for all teachings related to formulations for an APC. Additional methods of formulation may be found in U.S. Pat. Nos. 8,232,315; 8,440,715; 8,552,060; 8,623,913; 8,729,120; 8,741,950; 8,877,806; 8,895,609; 8,927,602; 9,226,910; 9,359,290; 9,403,761; 9,604,917; 9,610,274; 9,907,777; 10,351,517; and U.S. Publication Nos. 2015/0018414 and are herein incorporated by reference.

### V. Methods of Treatment and Use of APC

In some embodiments, an APC may ameliorate biomarkers for cardiovascular disease in addition to being used for treatment of another disease or disorder. In some embodiments, a patient that is treated with an APC may have a BMI outside of the normal range and also have another disease or disorder. Methods of treatment include those found in U.S. Pat. Nos. 8,232,315; 8,440,715; 8,552,060; 8,623,913; 8,729,120; 8,741,950; 8,877,806; 8,895,609; 8,927,602; 9,226,910; 9,359,290; 9,403,761; 9,604,917; 9,610,274; 9,907,777; 10,195,151; 10,351,517; 10,512,609; and U.S. Publication Nos. 2015/0018414 and 2020/0085748 and are incorporated by reference herein for this purpose.

### VI. Dosing of an APC

In some embodiments, the present disclosure provides methods for dosing of (R)-2-amino-3-phenylpropylcarbamate (APC). In some embodiments, the patient receives about 75-150 mg/day of (R)-2-amino-3-phenylpropylcarbamate (APC). In some embodiments, the patient receives about 37.5, 75, 150, or 300 mg/day of (R)-2-amino-3-phenylpropylcarbamate (APC).

The amounts of APC of the present disclosure that are to be delivered will depend on many factors, for example, the IC50, EC50, the biological half-life of the compound, the age, size, weight, and physical condition of the patient, and the disease or disorder to be treated. The importance of these and other factors to be considered are well known to those of skill in the art. Generally, the amount of APC of the present disclosure will be administered (also stated herein as “the patient receives”) at a range from about 1 milligram (mg) per day (mg/day) to 1,000 mg/day, with a dosage range of about 10 mg/day to about 800 mg/day to ameliorate biomarkers associated with cardiovascular risk. Of course, other dosages and/or treatment regimens may be employed, as determined by the attending physician.

In some embodiments, the method comprises administering APC of the present disclosure at an amount from about 10 mg/day to about 800 mg/day. In some embodiments, the method comprises administering APC of the present disclosure at an amount from 10 mg/day to 800 mg/day. In some embodiments, APC of the present disclosure is administered in an amount of about 10, 25, 37.5, 50, 75, 100, 125, 150, 200, 300, 400, 500, 600, 700, or 800 mg/day or an equivalent amount thereof (for example on a protein content basis). In a more specific embodiment, APC of the present disclosure is administered at an amount selected from the group consisting of about 10, 25, 37.5, 50, 75, 100, 125, 150, 200, 300, 400, 500, 600, 700, and about 800 mg/day. In a more specific embodiment, APC of the present disclosure is administered at an amount selected from the group consisting of 10, 25, 37.5, 50, 75, 100, 125, 150, 200, 300, 400, 500, 600, 700, and 800 mg/day. In another specific embodiment, APC of the present disclosure is administered at a dose more than or equal to about 10, 25, 37.5, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, or 800 mg/day. In another specific embodiment, APC of the present disclosure is administered at a dose less than or equal to about 800, 775, 750, 725, 700, 675, 650, 625, 600, 575, 550, 525, 500, 475, 450, 425, 400, 375, 350, 325, 300, 275, 250, 225, 200, 175, 150, 125, 100, 75, 50, 37.5 25, or 10 mg/day.

In another exemplary embodiment, APC of the present disclosure is administered in an amount between about 37.5 mg/day and about 300 mg/day. In another exemplary embodiment, APC of the present disclosure is administered in an amount between 37.5 mg/day and 300 mg/day. In some embodiments, APC of the present disclosure is administered in an amount of about 37.5, 50, 75, 100, 125, 150, 200, 225, 250, 275 or 300 mg/day or an equivalent amount thereof. In a more specific embodiment, APC of the present disclosure is administered at an amount selected from the group consisting of about 37.5, 50, 75, 100, 125, 150, 200, 225, 250, 275, and about 300 mg/day. In a more specific embodiment, APC of the present disclosure is administered at an amount selected from the group consisting of 37.5, 50, 75, 100, 125, 150, 200, 225, 250, 275, and 300 mg/day. In a more specific embodiment, APC of the present disclosure is administered at an amount selected from the group consisting of about 37.5, 75, 150, and about 300 mg/day. In a more specific embodiment, APC of the present disclosure is administered at an amount selected from the group consisting of 37.5, 75, 150, and 300 mg/day.

In an exemplary embodiment, APC of the present disclosure is administered in an amount between about 75 mg/day and about 150 mg/day. In another exemplary embodiment, APC of the present disclosure is administered in an amount between 75 mg/day and 150 mg/day. In some embodiments, APC of the present disclosure is administered in an amount of about 75, 80, 85, 90, 95, 100, 105, 110, 115, 120, 125, 130, 135, 140, 145, or 150 mg/day or an equivalent amount thereof. In a more specific embodiment, APC of the present disclosure is administered at an amount selected from the group consisting of about 75, 80, 85, 90, 95, 100, 105, 110, 115, 120, 125, 130, 135, 140, 145, and about 150 mg/day. In a more specific embodiment, APC of the present disclosure is administered at an amount selected from the group consisting of 75, 80, 85, 90, 95, 100, 105, 110, 115, 120, 125, 130, 135, 140, 145, and 150 mg/day.

In one embodiment of the invention, APC is administered to the subject as needed to treat a disorder. The compound can be administered continuously or intermittently. In one embodiment, the compound is administered to the subject more than once a day, e.g., 2, 3, or 4 times per day, or once every 1, 2, 3, 4, 5, 6, or 7 days. In another embodiment, the compound is administered to the subject no more than once a week, e.g., no more than once every two weeks, once a month, once every two months, once every three months, once every four months, once every five months, once every six months, or longer. In a further embodiment, the compound is administered using two or more different schedules, e.g., more frequently initially (for example to build up to a certain level, e.g., once a day or more) and then less frequently (e.g., once a week or less). In other embodiments, the compound can be administered by any discontinuous administration regimen. In one example, the compound can be administered not more than once every three days, every four days, every five days, every six days, every seven days, every eight days, every nine days, or every ten days, or longer. The administration can continue for one, two, three, or four weeks or one, two, or three months, or longer. Optionally, after a period of rest, the compound can be administered under the same or a different schedule. The period of rest can be one, two, three, or four weeks, or longer, according to the pharmacodynamic effects of the compound on the subject. In another embodiment the compound can be administered to build up to a certain level, then maintained at a constant level and then a tailing dosage. In some embodiments, the dosage may be increased based on efficacy and tolerability. In some embodiments, the dosage may be doubled at intervals of at least three days based on efficacy and tolerability.

In some embodiments, duration of treatment with an APC can last for at least 52 weeks. In some embodiments, duration of treatment with an APC can last for at least 50 weeks. In some embodiments, duration of treatment with an APC can last for at least 48 weeks. In some embodiments, duration of treatment with an APC can last for at least 44 weeks. In some embodiments, duration of treatment with an APC can last for at least 42 weeks. In some embodiments, duration of treatment with an APC can last for at least 41 weeks. In some embodiments, duration of treatment with an APC can last for at least 40 weeks. In some embodiments, the duration of treatment with an APC can last for 40 weeks. In some embodiments, the duration of treatment with an APC can last for at least 39 weeks. In some embodiments the duration of treatment with an APC can last for at least 38 weeks. In some embodiments, the duration of treatment with an APC can last for at least 37 weeks. In some embodiments, the duration of treatment with an APC can last for at least 36 weeks. In some embodiments, the duration of treatment with an APC can last for at least 30 weeks. In some embodiments, duration of treatment with an APC can last for at least 26 weeks. In some embodiments, the duration of treatment with an APC can last for at least 24 weeks. In some embodiments, the duration of treatment with an APC can last for at least 20 weeks. In some embodiments, the duration of treatment with an APC can last for at least 16 weeks. In some embodiments, the duration of treatment with an APC can last for at least 12 weeks. In some embodiments, the duration of treatment with an APC can last for at least 8 weeks. In some embodiments, the duration of treatment with an APC can last for at least 4 weeks. In some embodiments, the duration of treatment with an APC can last for at least 2 weeks. In some embodiments, the duration of treatment with an APC can last for at least 1 week.

Other possible dosing regimens for an APC or related compounds can be found in U.S. Pat. Nos. 8,232,315; 8,440,715; 8,552,060; 8,623,913; 8,729,120; 8,741,950; 8,877,806; 8,895,609; 8,927,602; 9,226,910; 9,359,290; 9,403,761; 9,604,917; 9,610,274; 9,907,777; 10,195,151; 10,351,517; 10,512,609; and U.S. Publication Nos. 2015/0018414 and 2020/0085748 and are incorporated by reference herein for these purposes.

In one aspect of the invention, APC is delivered to a subject concurrently with an additional therapeutic agent. The additional therapeutic agent can be delivered in the same composition as the compound or in a separate composition. The additional therapeutic agent can be delivered to the subject on a different schedule or by a different route as compared to the compound. The additional therapeutic agent can be any agent that provides a benefit to the subject. Further agents include, without limitation, stimulants, anti-psychotics, anti-depressants, agents for neurological disorders, and chemotherapeutic agents. One therapeutic agent that can be administered during the same period is Xyrem®, sold commercially by Jazz Pharmaceuticals, which is used to treat narcolepsy and cataplexy. See U.S. Pat. Nos. 8,952,062 and 9,050,302.

### EXAMPLES

### Example 1: Study to Evaluate Toxicity and Toxicokinetics

A 1-year open-label extension (OLE) study was performed with 374 participants to examined whether clinically significant weight loss (≥5%) in this population had favorable effects on biomarkers of cardiovascular risk compared to no such weight loss. The study measured changes in weight and BP (parent study baseline to OLE week 40) and clinical laboratory assessments (OLE baseline to week 40) in participants with narcolepsy (n=124) or OSA (n=250) who received solriamfetol at doses of 75, 150, or 300 mg/d.

Results from the study showed ≥5% weight loss in 4.5%, 17.3%, and 32.4% of participants with narcolepsy or OSA receiving solriamfetol 75, 150, or 300 mg/d. Of 374 participants, 96 (25.7%) had ≥5% weight loss; among those without ≥5% weight loss (n=278), 34 (9.1% of total population) had ≥5% weight gain. Demographics were similar in those with and without weight loss (see Table 1 (demographics and baseline characteristics) and Table 2 (additional demographics and baseline characteristics)). From baseline to week 40, there were decreases in percentage of participants with high serum glucose (36.6% to 28.1%) or high triglycerides (26.6% to 21.9%) among participants with weight loss versus increased glucose (43.3% to 50%) and no change in triglycerides (37.1% to 37.2%) in participants without weight loss (see Table 6 (lipid maximum changes from baseline to week 40), Table 7 (glucose changes from baseline to week 40), Table 9 (triglycerides categories at baseline to week 40), Table 10 (glucose categories at baseline to week 40)). The percentage of participants with high total cholesterol did not change among participants with weight loss (22.3% to 22.9%) and increased (32.7% to 37.2%) in participants without weight loss (see Table 6 and Table 8 (total cholesterol categories at baseline to week 40)). Participants with weight loss had mean±SD reductions in SBP (−2.6±11.4 mmHg) and DBP (−1.0±9.0 mmHg), whereas participants without weight loss had increases of +0.65±12.5 mmHg and +1.2±8.7 mmHg, respectively (see Table 3 (blood pressure at baseline and week 40), Table 4 (systolic blood pressure (SBP) maximum changes from baseline to week 40), and Table 5 (diastolic blood pressure (DBP) maximum changes from baseline to week 40)). Adverse events are shown in Table 11 (adverse events in ≥5% of Combined Solriamfetol Participants).

Among solriamfetol-treated participants who experienced ≥5% weight loss, there were decreases in BP and percentage of participants with high glucose and triglycerides. Further research is required to examine prospective long-term effects of solriamfetol on specific biomarkers of cardiovascular risk.

### Example 2: Effects of Solriamfetol on 24-Hour Blood Pressure Patterns in Participants with Excessive Daytime Sleepiness Associated With Narcolepsy

Solriamfetol is a dopamine and norepinephrine reuptake inhibitor indicated to improve wakefulness in adult patients with excessive daytime sleepiness associated with narcolepsy (75-150 mg/d) or obstructive sleep apnea (37.5-150 mg/d). Previous studies reported small mean increases in blood pressure (BP); however, the time course of these effects has not been evaluated. In addition, effects on BP dipping, which has been shown to be a risk factor for adverse cardiovascular outcomes, have not been evaluated. These analyses evaluated the effects of solriamfetol treatment on BP using 24-hour ambulatory blood pressure monitoring (ABPM) and on the percentage of narcolepsy patients with a non-dipping BP profile.

Twenty-four-hour ABPM was conducted at baseline and week 8 in a 12-week randomized controlled trial in participants with narcolepsy (n=236). Blood pressure was collected every 30 minutes.

Baseline demographics and clinical characteristics of the study population were representative of the real-world narcolepsy population, with mean body mass index in the overweight range and hypertension in approximately 20% of participants (see Table 12).

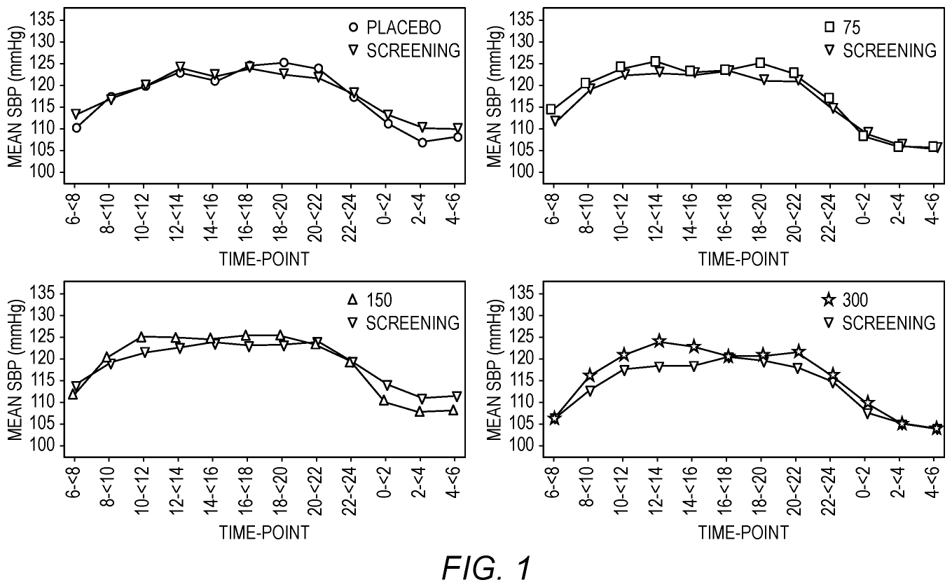

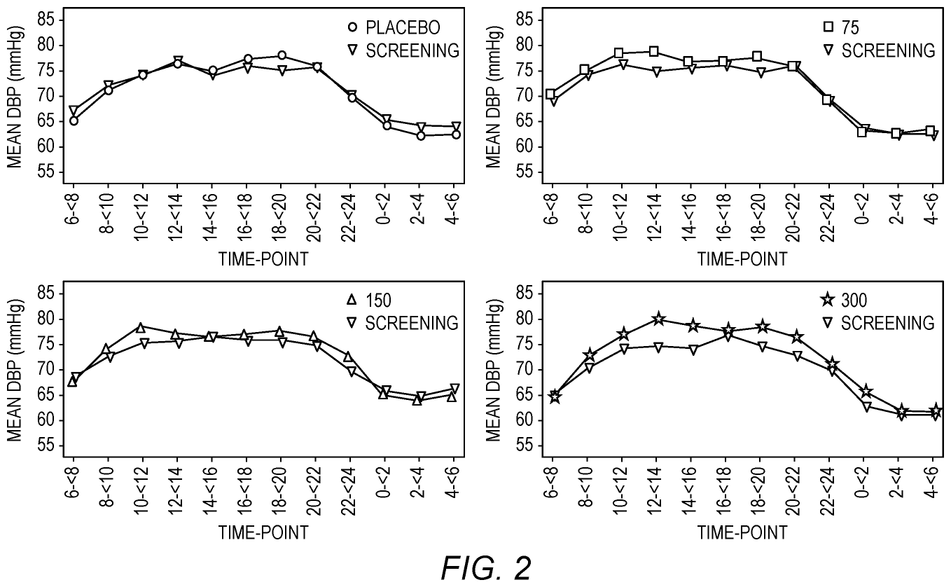

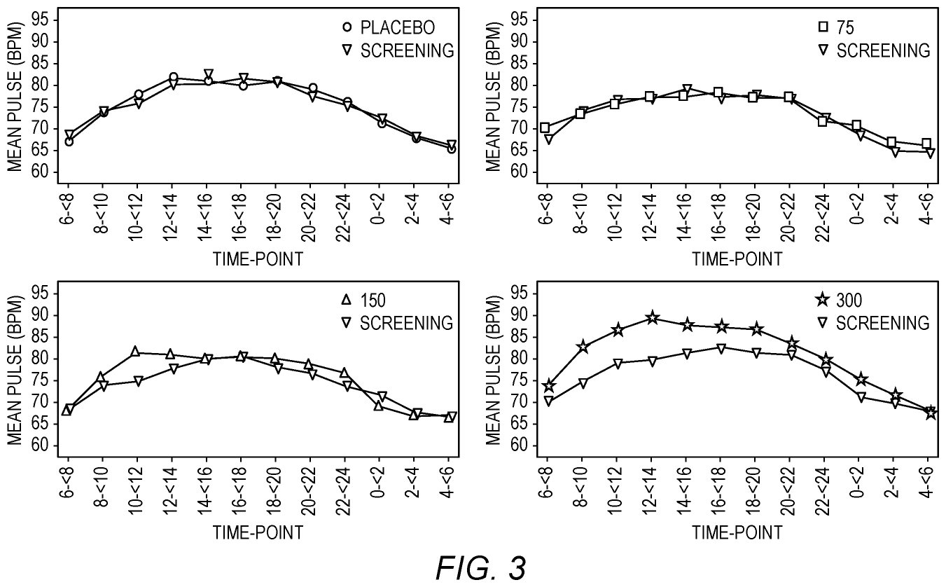

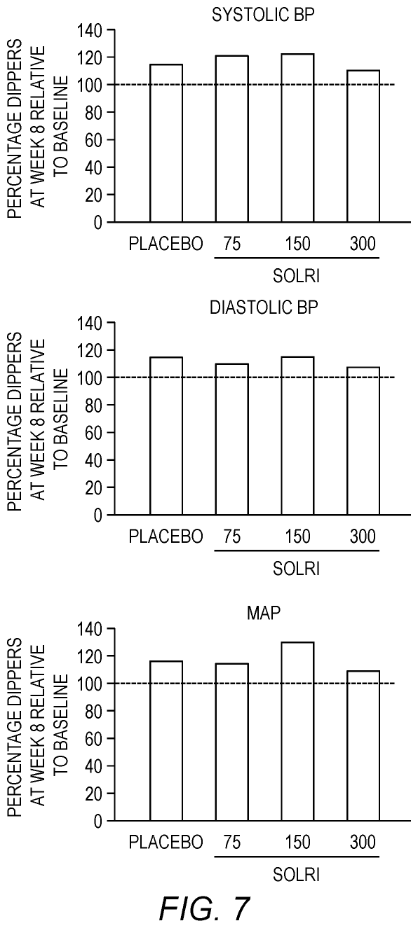

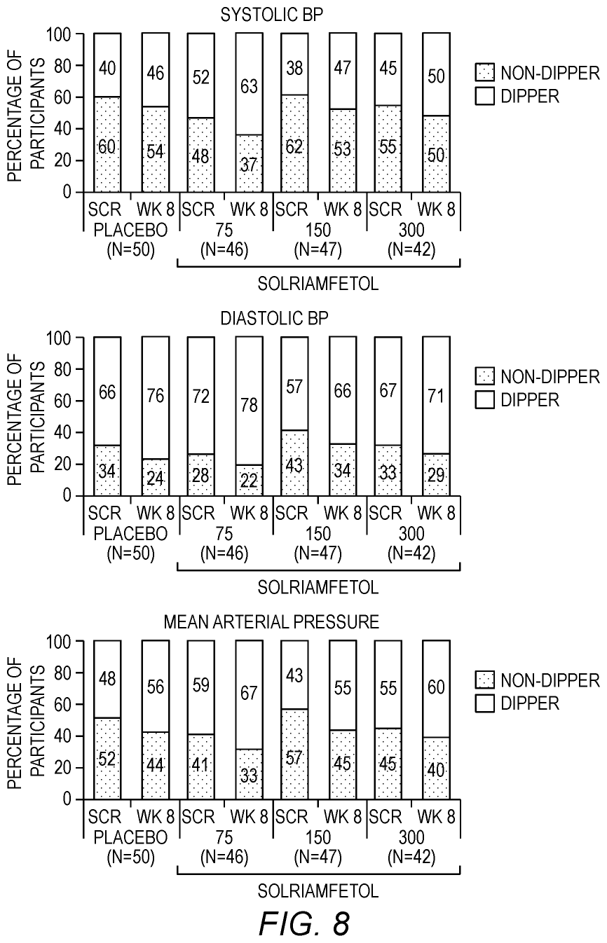

At week 8, increases in BP were apparent in the 150 and 300 mg dose groups from 8 AM until 4 PM and 6 PM, respectively (see Table 13 (Ambulatory Blood Pressure Monitoring (ABPM) vital sign data: Mean Change in Vital Signs on ABPM from Screening to Week 8) and FIGS. 1-3). Mean changes in SBP from screening to week 8 were −0.3, 1.8, −0.5, and 2.4 mmHg for the placebo, solriamfetol 75-, 150-, and 300-mg dose groups, respectively. At week 8, increases in SBP were transient throughout the day. Mean changes in DBP from screening to week 8 were −0.1, 1.4, 0.4, and 3.0 mmHg for the placebo, solriamfetol 75-, 150-, and 300-mg dose groups, respectively. At week 8, increases in DBP were transient throughout the day At baseline, 52% (placebo) and 48% (combined solriamfetol) of participants were non-dippers (defined as <10% decrease in mean arterial pressure [MAP] during sleep). There was no increase in the percentage of non-dippers at week 8 relative to baseline (placebo, 44%; combined solriamfetol, 39%) (see FIGS. 7 and 8). Results were similar when dipping was defined by changes in systolic BP and diastolic BP.

The effects of solriamfetol on BP at the highest approved dose of 150 mg/d are transient across the day. Solriamfetol was not observed to have an increase in non-dipping classification in participants with narcolepsy at any dose studied.

### Example 3: Effects of Solriamfetol on 24-Hour Blood Pressure Patterns in Participants with Excessive Daytime Sleepiness Associated With Obstructive Sleep Apnea

Solriamfetol is a dopamine and norepinephrine reuptake inhibitor indicated to improve wakefulness in adult patients with excessive daytime sleepiness associated with obstructive sleep apnea (OSA; 37.5-150 mg/d) or narcolepsy (75-150 mg/d). Previous studies reported small mean increases in blood pressure (BP); however, the time course of these effects has not been evaluated. In addition, effects on BP dipping, which has been shown to be a risk factor for adverse cardiovascular outcomes have not been evaluated. These analyses evaluated the effects of solriamfetol treatment on BP using 24-hour ambulatory blood pressure monitoring (ABPM) and on the percentage of OSA patients with a non-dipping BP profile.

Twenty-four-hour ABPM was conducted at baseline and week 8 in a 12-week randomized controlled trial in participants with OSA (n=474). Blood pressure was collected every 30 minutes.

Baseline demographics and clinical characteristics of the study population were representative of the real-world OSA population, with mean body mass index in the obese range and the majority of participants being male and hypertensive (see Table 14 (demographic and baseline clinical characteristics)).

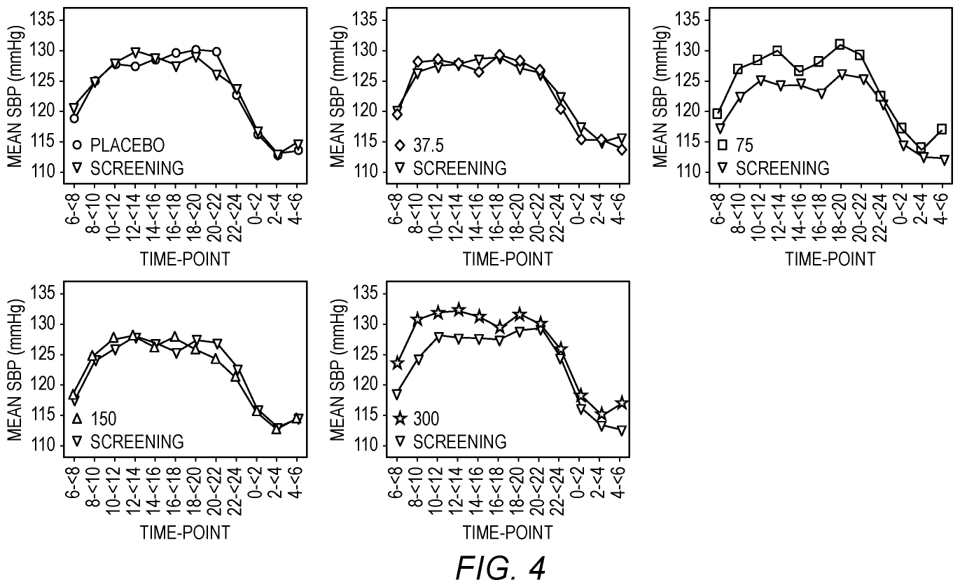

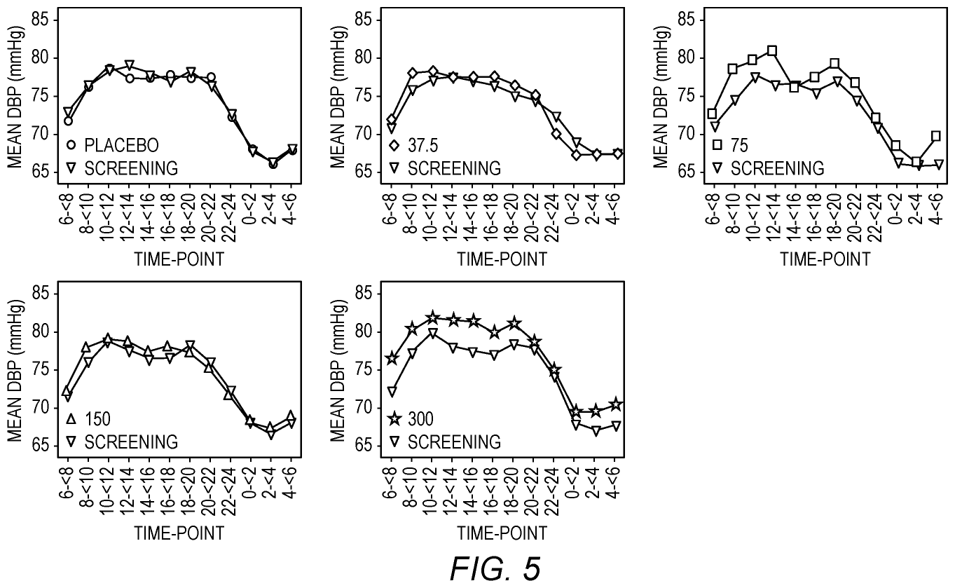

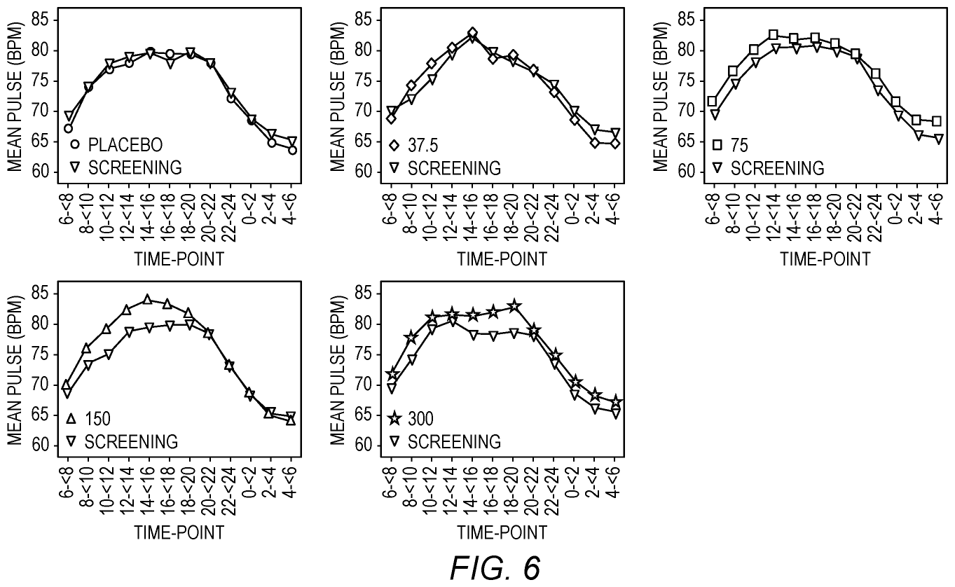

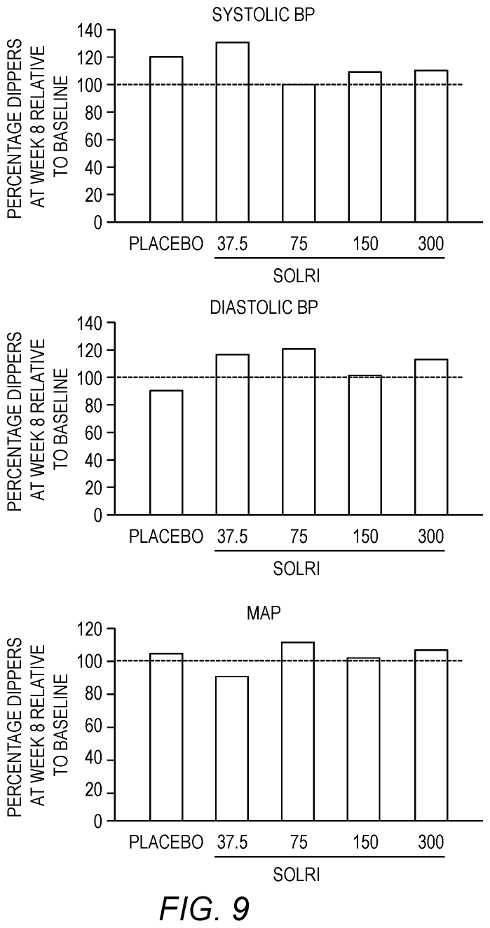

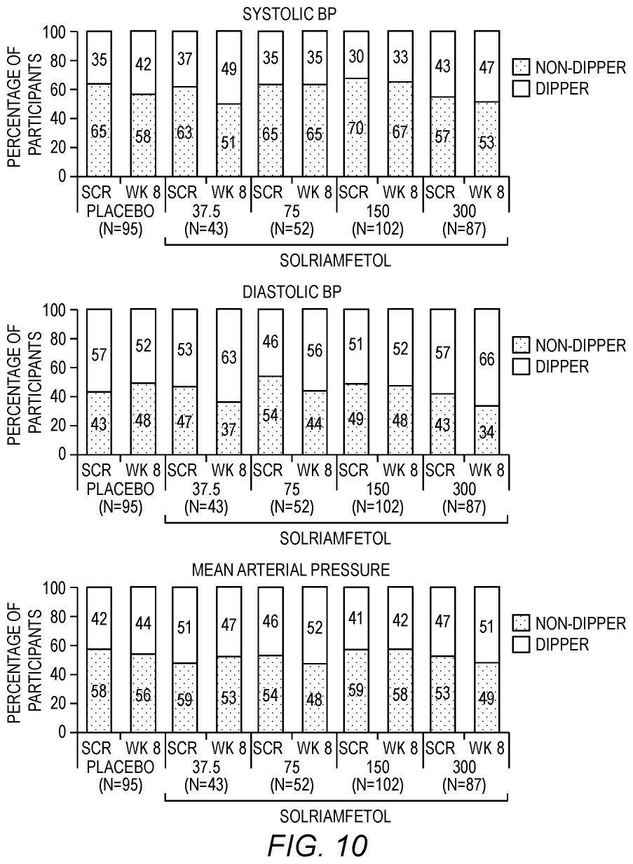

At week 8, increases in BP were apparent in the 75 and 300 mg dose groups from ˜6 AM until 8 PM (see Table 15 (ABPM vital sign data for Study 14-003, 12-week, double-blind, randomized, placebo-controlled study: Mean Change in Vital Signs on ABPM from Screening to Week 8) and FIGS. 4-6). Mean changes in SBP from screening to week 8 were −0.8, 0.7, 2.2, −0.5, and 2.9 mmHg for the placebo, solriamfetol 37.5-, 75-, 150-, and 300-mg dose groups, respectively. At week 8, increases in SBP were transient throughout the day. Mean changes in DBP from screening to week 8 were −0.1, 0.8, 0.7, −0.2, and 2.5 mmHg for the placebo, solriamfetol 37.5-, 75-, 150-, and 300-mg dose groups, respectively. At week 8, increases in DBP were transient throughout the day. At baseline, 58% (placebo) and 55% (combined solriamfetol) of participants were non-dippers (defined as <10% decrease in mean arterial pressure [MAP] during sleep). There was no increase in the percentage of non-dippers at week 8 relative to baseline (placebo, 56%; combined solriamfetol, 53%) (see FIGS. 9 and 10). Results were similar when dipping was defined by changes in systolic BP and diastolic BP.

The effects of solriamfetol on BP at the highest approved dose of 150 mg/d are transient across the day. Solriamfetol was not observed to have an increase in non-dipping classification in participants with OSA at any dose studied.

### Example 4: Effects of Weight Loss During Long-Term Solriamfetol Treatment on Cardiometabolic Indices

Increased prevalence of obesity has been reported in patients with narcolepsy and obstructive sleep apnea (OSA). Results from a 1-year open-label extension (OLE) study showed ≥5% weight loss in 4.5%, 17.3%, and 32.4% of participants with narcolepsy or OSA receiving solriamfetol 75, 150, or 300 mg/d. We examined whether clinically significant weight loss (≥5%) in this population had favorable effects on biomarkers of cardiovascular risk compared to no such weight loss.

We evaluated changes in weight and BP (parent study baseline to OLE week 40) and clinical laboratory assessments (OLE baseline to week 40) in participants with narcolepsy (n=124) or OSA (n=250) from the OLE study.

Of 374 participants, 96 (25.7%) had ≥5% weight loss; among those without ≥5% weight loss (n=278), 34 (9.1% of total population) had ≥5% weight gain. Demographics were similar in those with and without weight loss. From baseline to week 40, there were decreases in percentage of participants with high serum glucose (36.6% to 28.1%) or high triglycerides (26.6% to 21.9%) among participants with weight loss versus increased glucose (43.3% to 50%) and no change in triglycerides (37.1% to 37.2%) in participants without weight loss. The percentage of participants with high total cholesterol did not change among participants with weight loss (22.3% to 22.9%) and increased (32.7% to 37.2%) in participants without weight loss. Participants with weight loss had mean±SD reductions in SBP (−2.6±11.4 mmHg) and DBP (−1.0±9.0 mmHg), whereas participants without weight loss had increases of +0.65±12.5 mmHg and +1.2±8.7 mmHg, respectively.

Among solriamfetol-treated participants who experienced ≥5% weight loss, there were decreases in BP and percentage of participants with high glucose and triglycerides. Further research is required to examine prospective long-term effects of solriamfetol on specific biomarkers of cardiovascular risk.

### Example 5: Cardiovascular Effects During Solriamfetol Treatment in Phase 3 Trials in Obstructive Sleep Apnea

Obstructive sleep apnea (OSA) is associated with a high rate of cardiovascular (CV) comorbidities. Solriamfetol, a dopamine/norepinephrine reuptake inhibitor, is approved in the US and EU to treat excessive daytime sleepiness (EDS) associated with OSA (37.5 to 150 mg/d) or narcolepsy (75 to 150 mg/d). Small, dose-related mean changes in blood pressure (BP) and heart rate (HR) have been observed with solriamfetol treatment in clinical trials. In the 12-week trial, mean changes from baseline to week 12, averaged across predose to 10 hours postdose for all solriamfetol dose groups, ranged from 0.5 to 2.5 mm Hg for systolic BP (SBP), −0.2 to 1.5 mm Hg for diastolic BP (DBP), and 0.7 to 2.9 bpm for HR, with the greatest changes observed with 300 mg; changes with placebo were minimal (−0.2 mm Hg, 0.0 mm Hg, and 0.1 bpm, respectively). In the OLE, minimal changes from baseline to week 40 were observed (SBP, −0.49 mm Hg; DBP, 0.29 mm Hg; HR, −1.94 bpm).

We evaluated clinical CV outcomes associated with solriamfetol in participants with OSA from phase 3 clinical trials.

An exploratory analysis of data from one 12-week randomized, controlled trial (Schweitzer P K, et al. Am J Respir Crit Care Med. 2019; 199(11):1421-31) and 1 long-term open-label extension (OLE) trial in adults with EDS (Epworth Sleepiness Scale score ≥10) associated with OSA (Malhotra A, et al. Sleep. 2020; 43(2):zsz220) was carried out. Participants with current/prior use of primary OSA therapy (positive airway pressure [PAP], oral pressure therapy, oral appliance, or upper airway stimulation) were eligible, regardless of the level of adherence to primary OSA therapy; participants who had not attempted to use primary OSA therapy were excluded. Adherence to primary OSA therapy was defined as PAP use ≥4 hours/night on ≥70% of nights/week, historical report (with investigator concurrence) of oral appliance use on ≥70% of nights, or effective surgical intervention. Nonadherence to primary OSA therapy was defined as device use at a level lower than specific above, no device use at all, or surgical intervention deemed no longer effective. Participants received placebo or solriamfetol 37.5 (12-week trial only), 75, 150, or 300 mg/day for 12 weeks (12-week trial) or ≤40 weeks (OLE trial). Vital signs, antihypertensive medication use, and adverse events (AEs) were monitored throughout the studies. Baseline demographics, CV comorbidities, and concomitant medications, the percentage of participants who initiated antihypertensive medication use during treatment, and the incidence of and discontinuations due to AEs of hypertension/increased BP were summarized for the safety population (participants who received ≥1 dose of study medication).

In the 12-week trial, 51.3% of participants on placebo and 49.0% on solriamfetol (all doses) were on antihypertensive medication at baseline (see Table 16). In the OLE trial, 51.3% of participants were on antihypertensive medication at baseline. The data show few solriamfetol-treated participants initiated antihypertensive medication during either trial. Medication includes use of antihypertensive medication for any indication; first dose of antihypertensive medication occurred after first dose of study medication.

Major demographic factors and baseline CV characteristics were generally similar between the trial populations and real-world populations (see Table 17). Apnea-hypopnea index values were lower in the solriamfetol trial populations because many of the participants were using primary OSA therapy. At baseline, the majority of participants were adherent to primary OSA therapy. In the 12-week trial, 334 (70.5%) were adherent and 140 (29.5%) were nonadherent. In the OLE, 255 (76.6%) participants were adherent and 78 (23.4%) were nonadherent. The data show the trial populations were representative of the real-world OSA population. In solriamfetol studies, hypertension represents clustered preferred terms of hypertension, essential hypertension, and prehypertension. Hyperlipidemia represents clustered preferred terms of hyperlipidemia, hypercholesterolemia, blood cholesterol increased, hypertriglyceridemia, dyslipidemia, high-density lipoprotein decreased, and type V hyperlipidemia. Diabetes mellitus represents clustered preferred terms of type 2 diabetes mellitus, diabetes mellitus, glucose tolerance impaired, hyperglycemia, blood glucose increased, impaired fasting glucose, glucose urine present, hyperinsulinemia, and insulin resistance.

In the 12-week study, a total of 10 participants had AEs of hypertension/increased BP (see Table 18). The data show few solriamfetol-treated participants discontinued due to adverse events of hypertension/increased BP during either study. BP includes AE preferred terms of blood pressure increased, hypertension, and procedural hypertension. Adverse events coded using (MedDRA) Version 18.0. Five of the 10 participants had a history of hypertension and 1 required modification of their current antihypertensive therapy and none required initiation of antihypertensive medication. In the OLE, a total of 22 participants had AEs of hypertension/increased BP. Thirteen of the 22 participants had a history of hypertension and required anti-HTN medication or modification of their current antihypertensive therapy. No AEs of hypertension/increased BP were serious; most were mild or moderate in severity, and the majority resolved over time with continued solriamfetol dosing. In the 12-week trial, there were no serious CV AEs. In the OLE trial, there was 1 serious AE of cerebrovascular accident. In the OLE trial, AEs of hypertension/increased BP appeared to be dose-dependent, which is more likely to reflect what will be observed in clinical practice where there is a clinical titration to affect. Also, in clinical practice, the starting dose will be lower (37.5 mg/d) and the dose can be decreased in response to an AE. The majority of participants with these AEs had a history of hypertension and were treated with antihypertensive medication prior to AE onset.

Baseline characteristics and concomitant medication use of the study population were reflective of the real-world OSA population. At baseline, approximately half of the OSA study population had hypertension and/or used antihypertensive medications. Although small dose-related mean changes in BP and HR were observed during the 12-week and long-term OLE trials, few participants initiated new antihypertensive medication or discontinued due to AEs of hypertension or increased BP. The recommended starting dose of solriamfetol in patients with OSA is 37.5 mg/d; careful titration, appropriate patient selection, and routine monitoring of vital signs during treatment is recommended.

### Example 6: A 52-Week, Randomized, Double-blind, Placebo-Controlled, Parallel-Group Study to Evaluate Effect of Solriamfetol on Weight Loss in Overweight or Obese OSA Subjects with Excessive Daytime Sleepiness

This is a multicenter, randomized, double-blind, placebo-controlled, parallel-group study to evaluate the effect of solriamfetol in weight reduction and the associated reduction in CV risk in overweight or obese OSA patients with excessive daytime sleepiness (EDS) who are looking to lose weight. After screening for eligibility subjects will be randomly assigned in a 1:1 ratio to solriamfetol or placebo. Study visits will occur at 2 weeks after randomization and monthly thereafter. At week 26 subjects who were initially randomized to receive placebo will switch to solriamfetol treatment and those who were randomized to solriamfetol will continue on the same treatment for the remainder of the study. A safety follow-up call will be made 7-14 days after the completion of the 52-week treatment. All subjects will receive nutrition and exercise counseling at baseline and support throughout the study. Subjects will keep a food and exercise diary to assist with counseling support. Subjects who choose to discontinue study medication are encouraged to return for study visits through the 12-month study period.

### Primary Objective:

To assess the effects of solriamfetol in weight loss

### Secondary Objectives:

To assess any changes in cardiovascular risk factors associated with obesity (i.e., dyslipidemia, insulin sensitivity, hypertension, central fat distribution, biomarkers of CV risk)

To assess the ongoing safety of solriamfetol

To assess changes in Quality of Life measures

### Inclusion Criteria:

Each subject must meet the following criteria to be enrolled in the study: (1) Male or female between 18 and 75 years of age, inclusive, (2) Diagnosis of OSA according to ICSD-3 criteria, (3) BMI 27-45 kg/m², (4) Baseline Epworth Sleepiness Scale (ESS) score ≥10, (5) Usual nightly total sleep time of at least 6 hours, (7) Consent to use a medically acceptable method of contraception for at least 2 months prior to the first dose of study drug, throughout the entire study period, and for 30 days after the study is completed, (8) Ambulatory and able to perform moderate exercise, (9) Willing and able to comply with the study design schedule and other requirements, including instructions from nutritional and exercise counseling, (10) Willing and able to provide written informed consent Exclusion Criteria:

Subjects who demonstrate any of the following will be excluded from the study: (1) Female subjects who are pregnant, nursing, or lactating, (2) Have refused the use of CPAP or oral appliance, (3) Occupation requiring nighttime shift work or variable shift work, (4) Any other clinically relevant medical, behavioral (i.e. insufficient sleep), or psychiatric disorder other than OSA that is associated with excessive sleepiness, (5) Presence of uncontrolled hypertension, unstable cardiovascular or cerebrovascular disease, serious heart arrhythmias, or other serious heart problems that in the investigator's opinion may jeopardize subject safety in the study, (6) History or presence of any acutely unstable medical condition, behavioral or psychiatric disorder (including active suicidal ideation), or surgical history that could affect the safety of the subject or interfere with study efficacy, safety, or the ability of the subject to complete the trial per the judgment of the Investigator, (7) History of bariatric surgery or a history of roux-en-y procedure, (8) Current or recent history (past×years) of eating disorders (e.g., binge eating disorder), (9) Any weight change of 5 kg (11 pounds) in the last 3 months, (10) Presence of renal impairment or calculated creatinine clearance <60 mL/min, HbA1c>10% or laboratory values(s) outside the reference range that are considered to be clinically significant by the investigator (clinical chemistry, hematology, TSH, and urinalysis); Note: screening labs may be repeated once, (11) Clinically significant ECG abnormality in the opinion of the Investigator, (12) Treatment with insulin in any form or any GLP-1 inhibitor or pramlintide within 3 months prior to screening. Treatment with metformin, sulfonylurea, or combination with other oral medications such as DDP-IV inhibitors, meglitinides or acarbose are allowed and must be at a stable dose for at least 3 months prior to screening. Treatment with Thiazolidinediones (TZDs) is allowed and must be stable for at least 6 months prior to screening. (13) Treatment with over-the-counter weight loss products or appetite suppressants (including herbal weight loss agents) within 1 month of the screening visit, or with a prescription anti-obesity drug (e.g., lorcaserin, naltrexone SR/bupropion SR, phentermine/topiramate, phentermine, orlistat), topiramate, or lipid dissolving injections within 3 months. (14) Use of any over-the-counter (OTC) or prescription medications that could affect the evaluation of excessive sleepiness within a time period prior to the Baseline Visit corresponding to at least five half-lives of the drug(s) or planned use of such drug(s) at some point throughout the duration of the study. Examples of excluded medications include OTC sleep aids or stimulants (e.g., pseudoephedrine), methylphenidate, amphetamines, modafinil, armodafinil. Medications should be discontinued such that the subject has returned to his/her baseline level of daytime sleepiness at least 7 days prior to the Baseline visit, in the opinion of the Investigator. (15) Use of a monoamine oxidase inhibitor (MAOI) in the past 14 days or five half-lives (whichever is longer) prior to the Baseline Visit, or plans to use an MAOI during the study. (16) Received an investigational drug in the past 30 days or five half-lives (whichever is longer) prior to the Baseline Visit, or plans to use an investigational drug (other than the study drug) during the study. (17) Current or past (within the past 2 years) diagnosis of a moderate or severe substance use disorder according to DSM-5 criteria. (18) Current, past (within the past 2 years), or seeking treatment for a substance related disorder. (19) Urine drug screen positive for an illicit drug of abuse (including cannabinoids) at screening or at any point throughout the duration of the study, except for a prescribed drug (e.g., amphetamine) at screening.

### Test Product, Dose and Mode of Administration:

Solriamfetol 37.5 mg, 75 mg and 150 mg over-encapsulated opaque gelatin capsules administered once daily.

### Reference Therapy:

Placebo tablets will also be over-encapsulated in opaque gelatin capsules that will be identical to those used for the active solriamfetol treatments. Mode of administration will be the same as for the test product.

### Duration of Treatment:

52 weeks

### Efficacy Assessments:

Primary endpoint is: Mean % weight change from baseline to week 26.

Secondary endpoints are: (1) Proportion of subjects with ≥5%, 10% and 15% weight reduction from baseline to week 26, (2) Mean % of weight change from baseline to week 52, (3) Proportion of subjects with ≥5%, 10% and 15% weight reduction from baseline to week 52, (4) Mean weight change from baseline to week 26, (5) Mean weight change from baseline to week 52, (6) Mean change in waist, hip and neck circumferences from baseline to 6 months of study, (7) Mean change in waist, hip and neck circumferences from baseline to end of study, (8) Mean change in BP and HR, (9) Mean and categorical changes from baseline to 6 months and end of study in the following lab parameters: HDL, LDL, Triglycerides, HbA1c, Fasting glucose, Fasting insulin, HOMA-IR, C-reactive protein, IWQOL-Lite total score

### Safety Assessments:

The following safety assessments will be performed during the study: ECGs (at Screening, 6 and 12 months), At every visit: Vital signs, Physical examinations, Clinical laboratory tests, Adverse events (AEs) (only AEs at safety follow-up visit), C-SSRS, CPAP compliance downloadable only, Concomitant medication use (e.g. antihypertensive medications)

### CV Risk Assessments:

Assessments at Baseline, 3, 6, 9 and 12 months: Weight; Waist, hip and neck circumferences; HbA1c; Fasting glucose; Fasting insulin; Triglycerides; LDL cholesterol; HDL cholesterol; hsCRP

### Additional Assessments:

### ESS (at Screening and Every Visit)

At Screening and every 3 months: Impact of Weight on Quality of Life (or other Q of L questionnaires); Control of eating questionnaire; VAS for Satiety; Food Craving Inventory Statistical Analysis:

An interim datacut will be conducted when all subjects complete the week 26 visit (6 months) and the primary efficacy analysis will be performed to compare the difference in % mean weight change from baseline to week 26 between placebo and solriamfetol treatment.

Randomization of the subjects with diabetes will be stratified by HbA1c (<or ≥9%) and anti-diabetic medications.

### Sample Size Consideration:

The sample size will be approximately 400 (TBD).

The data generated from the proposed study will inform future label enhancement and will allow us to engage in discussions with the regulatory agencies in order to update section 14 in the solriamfetol label (USA) as well as section 5 of the SmPC. Thus, after the study is completed, the team will review the data with the goal of regulatory interactions to discuss potential inclusion of these data in the Sunosi (EDS in Narcolepsy and OSA) label, which could lead to an sNDA and/or Type II Variation in the US and EU, respectively.

Data from this study will also be published to provide clinically relevant information to clinicians and physicians associated with solriamfetol use in patents with OSA and EDS.

In summary if successful, the proposed study could: 1) support regulatory discussions around possible label changes to include the data in the clinical trial section of the USPI and SmPC, 2) provide clinically meaningful data to increase scientific understanding of solriamfetol effects in weight loss and its potential impact on cardiovascular risk reduction in patients with EDS in OSA, 3) further differentiate solriamfetol from other stimulants or wake promoting agents used to treat EDS in OSA.

While embodiments and applications of the present invention have been described in some detail by way of illustration and example, it would be apparent to those of skill in the art that many additional modifications would be possible without departing from the inventive concepts contained herein. All references cited herein are hereby incorporated in their entirety.

TABLE 1

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

Age (years)

| | | | | | | | | |
|---|---|---|---|---|---|---|---|---|
| Mean(SD) | 73.0 | 50.7(14.9) | 46.6(16.3) | 47.8(16.1) | 56.4(13.3) | 52.0(13.2) | 46.5(13.6) | 49.4(13.8) |
| Median | 73.0 | 51.0 | 47.0 | 49.0 | 58.0 | 54.0 | 48.5 | 51.0 |
| Min, Max | 73.0, 73.0 | 21.0, 73.0 | 18.0, 72.0 | 18.0, 73.0 | 27.0, 75.0 | 21.0, 76.0 | 18.0, 74.0 | 18.0, 76.0 |
| Male, n | 1 (100) | 9 (40.9) | 35 (47.9) | 45 (46.9) | 14 (66.7) | 57 (54.3) | 78 (51.3) | 149(53.6) |
| (%) | | | | | | | | |

Race, n (%)

| | | | | | | | | |
|---|---|---|---|---|---|---|---|---|
| AI/AN | 0 | 0 | 0 | 0 | 0 | 1(1.0) | 0 | 1 (0.4) |
| Asian | 0 | 1 (4.5) | 1 (1.4) | 2 (2.1) | 1 (4.8) | 6 (5.7) | 2 (1.3) | 9 (3.2) |
| Black/AA | 0 | 4 (18.2) | 8 (11.0) | 12 (12.5) | 4 (19.0) | 11 (10.5) | 34 (22.4) | 49 (17.6) |
| NH/OPI | 0 | 0 | 0 | 0 | 0 | 1 (1.0) | 0 | 1 (0.4) |
| White | 1 (100) | 17 (77.3) | 63 (86.3) | 81 (84.4) | 16 (76.2) | 86 (81.9) | 114 (75.0) | 216 (77.7) |
| Multiple | 0 | 0 | 1 (1.4) | 1 (1.0) | 0 | 0 | 2 (1.3) | 2 (0.7) |

AA, African American;
AI/AN, American Indian or Alaska Native;
NH/OPI, Native Hawaiian or Other Pacific Islander;
SD, standard deviation

TABLE 2

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

Region, n (%)

| | | | | | | | | |
|---|---|---|---|---|---|---|---|---|
| North | 1(100) | 20(90.9) | 64(87.7) | 85(88.5) | 19(90.5) | 94(89.5) | 141(92.8) | 254(91.4) |
| America | | | | | | | | |
| Europe | 0 | 2(9.1) | 9(12.3) | 11(11.5) | 2(9.5) | 11(10.5) | 11(7.2) | 24(8.6) |

BMI (kg/m²)

| | | | | | | | | |
|---|---|---|---|---|---|---|---|---|
| Mean | 39.9 | 32.5(7.6) | 32.0(5.9) | 32.2(6.3) | 32.1(5.1) | 31.9(5.7) | 31.5(6.2) | 31.7(5.9) |
| (SD) | | | | | | | | |
| Median | 39.9 | 33.8 | 32.4 | 32.9 | 32.8 | 32.4 | 31.3 | 31.9 |
| Min, Max | 39.9, 39.9 | 18.0, 45.0 | 21.3, 44.9 | 18.0, 45.0 | 21.8, 44.7 | 18.2, 44.7 | 18.0, 45.2 | 18.0, 45.2 |

BMI, body mass index;
SD, standard deviation

TABLE 3

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

SBP (mmHg)
Baseline

| | | | | | | | | |
|---|---|---|---|---|---|---|---|---|
| Mean | 137.0 | 128.4 (13.5) | 126.5 (11.9) | 127.1 (12.2) | 128.0 (12.0) | 124.6 (13.3) | 126.9 (13.9) | 126.1 (13.6) |
| (SD) | | | | | | | | |

Change - Wk 40

| | | | | | | | | |
|---|---|---|---|---|---|---|---|---|
| Mean | −3.3 | −4.5 (13.8) | −2.0 (10.8) | −2.6 (11.4) | −3.0 (12.3) | 2.30 (12.1) | −0.37 (12.7) | 0.65 (12.5) |
| (SD) | | | | | | | | |
| Median | −3.3 | −2.8 | −1.8 | −2.8 | 0.50 | 2.00 | 0.17 | 0.50 |
| Min, Max | −3.3, −3.3 | −48.5, 21.5 | −24.3, 21.0 | −48.5, 21.5 | −18.3, 17.0 | −24.5, 45.0 | −39.5, 29.0 | −39.5, 45.0 |

DBP (mmHg)
Baseline

| | | | | | | | | |
|---|---|---|---|---|---|---|---|---|
| Mean | 77.5 | 78.2 (7.7) | 77.7 (8.1) | 77.8 (8.0) | 79.5 (8.5) | 76.3 (8.9) | 79.1 (8.4) | 78.1 (8.7) |
| (SD | | | | | | | | |

Change - Wk 40

| | | | | | | | | |
|---|---|---|---|---|---|---|---|---|
| Mean | −3.8 | −1.9 (9.9) | −0.8 (8.9) | −1.0 (9.0) | 0.2 (9.5) | 2.3 (7.9) | 0.6 (9.1) | 1.2 (8.7) |
| (SD) | | | | | | | | |
| Median | −3.8 | 0.5 | −0.3 | −0.3 | 0.0 | 2.3 | 1.5 | 1.4 |
| Min, Max | −3.8, −3.8 | −22.5, 12.0 | −25.0, 18.7 | −25.0, 18.7 | −16.7, 17.0 | −20.0, 23.5 | −28.0, 21.0 | −28.0, 23.5 |

DBP, diastolic blood pressure;
SBP, systolic blood pressure;
SD, standard deviation

TABLE 4

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

SBP (mmHg), n (%)

| | | | | | | | | |
|---|---|---|---|---|---|---|---|---|
| Increase ≥10% | 0 | 3 (13.6) | 13 (17.8) | 16 (16.7) | 5 (23.8) | 26 (24.8) | 33 (21.7) | 64 (23.0) |
| Increase ≥20% | 0 | 1 (4.5) | 1 (1.4) | 2 (2.1) | 0 | 7 (6.7) | 8 (5.3) | 15 (5.4) |
| Increase ≥30% | 0 | 0 | 0 | 0 | 0 | 2 (1.9) | 0 | 2 (0.7) |
| Decrease ≥10% | 0 | 7 (31.8) | 20 (27.4) | 27 (28.1) | 6 (28.6) | 15 (14.3) | 36 (23.7) | 57 (20.5) |
| Decrease ≥20% | 0 | 1 (4.5) | 2 (2.7) | 3 (3.1) | 0 | 4 (3.8) | 10 (6.6) | 14 (5.0) |
| Decrease ≥30% | 0 | 1 (4.5) | 0 | 1 (1.0) | 0 | 0 | 2 (1.3) | 2 (0.7) |

TABLE 5

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

DBP (mmHg), n (%)

| | | | | | | | | |
|---|---|---|---|---|---|---|---|---|
| Increase ≥10% | 0 | 3 (13.6) | 8 (11.0) | 11 (11.5) | 4 (19.0) | 16 (15.2) | 21 (13.8) | 41 (14.7) |
| Increase ≥20% | 0 | 0 | 0 | 0 | 0 | 3 (2.9) | 2 (1.3) | 5 (1.8) |
| Increase ≥30% | 0 | 0 | 0 | 0 | 0 | 0 | 0 | 0 |
| Decrease ≥10% | 0 | 5 (22.7) | 11 (15.1) | 16 (16.7) | 3 (14.3) | 4 (3.8) | 20 (13.2) | 27 (9.7) |
| Decrease ≥20% | 0 | 1 (4.5) | 3 (4.1) | 4 (4.2) | 0 | 1 (1.0) | 5 (3.3) | 6 (2.2) |
| Decrease ≥30% | 0 | 0 | 0 | 0 | 0 | 0 | 0 | 0 |

TABLE 6

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

TC (mmol/L)
Baseline

| | | | | | | | | |
|---|---|---|---|---|---|---|---|---|
| n | 1 | 21 | 72 | 94 | 21 | 104 | 147 | 272 |
| Mean | 2.6 | 4.3 (0.8) | 4.5 (1.0) | 4.4 (1.0) | 5.0 (0.9) | 4.9 (1.1) | 4.7 (0.9) | 4.8 (1.0) |
| (SD) | | | | | | | | |

Change - Wk 40

| | | | | | | | | |
|---|---|---|---|---|---|---|---|---|
| n | 1 | 21 | 72 | 94 | 21 | 103 | 140 | 264 |
| Mean | 1.1 | 0.2 (0.5) | 0.2 (0.6) | 0.2 (0.6) | −0.1 (0.7) | 0.1 (0.7) | 0.1 (0.6) | 0.1 (0.6) |
| (SD) | | | | | | | | |
| Median | 1.1 | 0.2 | 0.2 | 0.2 | 0.2 | 0.1 | 0.100 | 0.100 |
| Min, Max | 1.1, 1.1 | −0.7, 1.5 | −1.3, 1.7 | −1.3, 1.7 | −1.7, 1.0 | −2.5, 2.1 | −2.1, 2.5 | −2.5, 2.5 |

TG (mmol/L)
Baseline

| | | | | | | | | |
|---|---|---|---|---|---|---|---|---|
| n | 1 | 21 | 72 | 94 | 21 | 104 | 147 | 272 |
| Mean | 1.4 | 1.4 (0.6) | 1.5 (1.0) | 1.5 (0.9) | 1.7 (0.7) | 1.8 (1.4) | 1.6 (0.9) | 1.7 (1.1) |
| (SD | | | | | | | | |

Change - Wk 40

| | | | | | | | | |
|---|---|---|---|---|---|---|---|---|
| n | 1 | 21 | 72 | 94 | 21 | 103 | 140 | 264 |
| Mean | 0.3 | −0.1 (0.4) | −0.3 (1.0) | −0.2 (0.9) | 0.2 (0.9) | −0.1 (1.0) | 0.0 (0.7) | −0.0 (0.9) |
| (SD) | | | | | | | | |
| Median | 0.3 | −0.2 | −0.2 | −0.2 | −0.1 | −0.0 | −0.1 | −0.0 |
| Min, Max | 0.3, 0.3 | −1.0, 0.9 | −6.1, 2.2 | −6.1, 2.2 | −1.1, 1.8 | −7.1, 3.7 | −3.0, 2.2 | −7.1, 3.7 |

SD is standard deviation;
TC is total cholesterol;
TG is triglycerides

TABLE 7

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

Glucose (mmol/L)
Baseline

| | | | | | | | | |
|---|---|---|---|---|---|---|---|---|
| n | 1 | 21 | 71 | 93 | 21 | 104 | 145 | 270 |
| Mean | 6.2 | 54 (0.8) | 5.7 (1.7) | 5.6 (1.6) | 6.9 (2.2) | 5.9 (1.6) | 5.9 (2.1) | 6.0 (1.9) |
| (SD) | | | | | | | | |

Change - Wk 40

| | | | | | | | | |
|---|---|---|---|---|---|---|---|---|
| n | 1 | 21 | 71 | 93 | 21 | 101 | 135 | 257 |
| Mean | −0.5 | −0.2 (0.8) | −0.2 (1.5) | −0.2 (1.4) | −0.1 (3.0) | −0.1 (1.1) | −0.1 (1.9) | −0.1 (1.8) |
| (SD) | | | | | | | | |
| Median | −0.5 | −0.2 | −0.1 | −0.1 | 0.2 | −0.1 | 0.0 | −0.0 |
| Min, Max | −0.5, −0.5 | −1.8, 1.1 | −8.5, 3.4 | −8.5, 3.4 | −7.7, 9.5 | −4.4, 2.9 | −8.8, 7.7 | −8.8, 9.5 |

SD, standard deviation

TABLE 8

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

Baseline, n (%)

| | | | | | | | | |
|---|---|---|---|---|---|---|---|---|
| n | 1 | 21 | 72 | 94 | 21 | 104 | 147 | 272 |
| Low | 0 | 0 | 0 | 0 | 0 | 0 | 0 | 0 |
| Normal | 1(100) | 17(81.0) | 55(76.4) | 73(77.7) | 11(52.4) | 66(63.5) | 106(72.1) | 183(67.3) |
| High | 0 | 4(19.0) | 17(23.6) | 21(22.3) | 10(47.6) | 38(36.5) | 41(27.9) | 89(32.7) |

Week 40, n (%)

| | | | | | | | | |
|---|---|---|---|---|---|---|---|---|
| n | 1 | 22 | 73 | 96 | 21 | 104 | 144 | 269 |
| Low | 0 | 0 | 0 | 0 | 0 | 0 | 0 | 0 |
| Normal | 1(100) | 18(81.8) | 55(75.3) | 74(77.1) | 13(61.9) | 58(55.8) | 98(68.1) | 169(62.8) |
| High | 0 | 4(18.2) | 18(24.7) | 22(22.9) | 8(38.1) | 46(44.2) | 46(31.9) | 100(37.2) |

TABLE 9

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

Baseline, n (%)

| | | | | | | | | |
|---|---|---|---|---|---|---|---|---|
| n | 1 | 21 | 72 | 94 | 21 | 104 | 147 | 272 |
| Low | 0 | 0 | 0 | 0 | 0 | 0 | 0 | 0 |
| Normal | 1(100) | 15(71.4) | 53(73.6) | 69(73.4) | 11(52.4) | 67(64.4) | 93(63.3) | 171(62.9) |
| High | 0 | 6(28.6) | 19(26.4) | 25(26.6) | 10(47.6) | 37(35.6) | 54(36.7) | 101(37.1) |

Week 40, n (%)

| | | | | | | | | |
|---|---|---|---|---|---|---|---|---|
| n | 1 | 22 | 73 | 96 | 21 | 104 | 144 | 269 |
| Low | 0 | 0 | 0 | 0 | 0 | 0 | 0 | 0 |
| Normal | 1(100) | 16(72.7) | 58(79.5) | 75(78.1) | 10(47.6) | 65(62.5) | 94(65.3) | 169(62.8) |
| High | 0 | 6(27.3) | 15(20.5) | 21(21.9) | 11(52.4) | 39(37.5) | 50(34.7) | 100(37.2) |

TABLE 10

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

Baseline, n (%)

| | | | | | | | | |
|---|---|---|---|---|---|---|---|---|
| n | 1 | 21 | 71 | 93 | 21 | 104 | 146 | 270 |
| Low | 0 | 0 | 2(2.8) | 2(2.2) | 0 | 0 | 1(0.7) | 1(0.4) |
| Normal | 0 | 13(61.9) | 44(62.0) | 57(61.3) | 6(28.6) | 57(54.8) | 89(61.4) | 152(56.3) |
| High | 1(100) | 8(38.1) | 25(35.2) | 34(36.6) | 15(71.4) | 47(45.2) | 55(37.9) | 117(43.3) |

Week 40, n (%)

| | | | | | | | | |
|---|---|---|---|---|---|---|---|---|
| n | 1 | 22 | 73 | 96 | 21 | 102 | 141 | 264 |
| Low | 0 | 0 | 0 | 0 | 0 | 0 | 0 | 0 |
| Normal | 0 | 15(68.2) | 54(74.0) | 69(71.9) | 5(23.8) | 55(53.9) | 72(51.1) | 132(50.0) |
| High | 1(100) | 7(31.8) | 19(26.0) | 27(28.1) | 16(76.2) | 47(46.1) | 69(48.9) | 132(50.0) |

TABLE 11

| Weight loss ≥5% at Week 40 (n = 96) | | | | Weight loss <5% at Week 40 (n = 278) | | | |
|---|---|---|---|---|---|---|---|
| Solri 75 | Solri 150 | Solri 300 | Solri - all | Solri 75 | Solri 150 | Solri 300 | Solri - all |
| (n = 1) | (n = 22) | (n = 73) | (n = 96) | (n = 21) | (n = 105) | (n = 152) | (n = 278) |

Common AEs, n(%)

| | | | | | | | | |
|---|---|---|---|---|---|---|---|---|
| Headache | 0 | 5(22.7) | 7(9.6) | 12(12.5) | 3(14.3) | 9(8.6) | 15(9.9) | 27(9.7) |
| Dry | 1(100) | 1(4.5) | 7(9.6) | 9(9.4) | 2(9.5) | 6(5.7) | 5(3.3) | 13(4.7) |
| mouth | | | | | | | | |
| Naso- | 0 | 0 | 8(11.0) | 8(8.3) | 3(14.3) | 14(13.3) | 15(9.9) | 32(11.5) |
| pharyn- | | | | | | | | |
| gitis | | | | | | | | |
| Insomnia | 0 | 2(9.1) | 5(6.8) | 7(7.3) | 4(19.0) | 14(13.3) | 3 (2.0) | 21(7.6) |
| Nausea | 0 | 4(18.2) | 3(4.1) | 7(7.3) | 0 | 12(11.4) | 8 (5.3) | 20(7.2) |
| Anxiety | 0 | 3(13.6) | 3(4.1) | 6(6.3) | 0 | 10(9.5) | 2(1.3) | 12(4.3) |
| Influenza | 0 | 1(4.5) | 5(6.8) | 6(6.3) | 0 | 3(2.9) | 2(1.3) | 5(1.8) |
| Sinusitis | 0 | 3(13.6) | 3(4.1) | 6(6.3) | 1(4.8) | 2(1.9) | 11(7.2) | 14(5.0) |
| Dizziness | 0 | 2(9.1) | 3(4.1) | 5(5.2) | 2(9.5) | 2(1.9) | 4(2.6) | 8(2.9) |
| URTI* | 0 | 1(4.5) | 3(4.1) | 4(4.2) | 0 | 6(5.7) | 14(9.2) | 20(7.2) |
| Feeling | 0 | 2(9.1) | 1(1.4) | 3(3.1) | 2(9.5) | 7(6.7) | 7(4.6) | 16(5.8) |
| jittery | | | | | | | | |

AE of interest, n(%)

| | | | | | | | | |
|---|---|---|---|---|---|---|---|---|
| Decreased | 0 | 0 | 2(2.7) | 2(2.1) | 1(4.8) | 5(4.8) | 6(3.9) | 12(4.3) |
| appetite | | | | | | | | |

*Upper Respiratory Tract Infection

TABLE 12

| | | | Solriamfetol | | | | | |
|---|---|---|---|---|---|---|---|---|
| | Placebo | | 75 mg | | 150 mg | | 300 mg | |
| | (N = 59) | | (N = 59) | | (N = 59) | | (N = 59) | |
| Age (years), mean (SD) | 36.0 | (15.2) | 36.5 | (12.8) | 38.1 | (13.0) | 34.3 | (11.5) |
| Male, n (%) | 24 | (40.7) | 22 | (37.3) | 17 | (28.8) | 19 | (32.2) |

Race, n (%)

| White | 47 | (79.7) | 46 | (78.0) | 48 | (81.4) | 48 | (81.4) |
|---|---|---|---|---|---|---|---|---|
| Black or African American | 10 | (16.9) | 12 | (20.3) | 6 | (10.2) | 5 | (8.5) |
| Asian | 0 | | 0 | | 3 | (5.1) | 3 | (5.1) |
| BMI (kg/m²), mean (SD) | 29.1 | (6.0) | 27.9 | (5.4) | 27.9 | (5.8) | 28.1 | (6.3) |
| MWT mean sleep latency | 6.1 | (5.6) | 7.5 | (5.4) | 7.7 | (5.6) | 8.7 | (6.2) |
| (min), mean (SD)^{a} | | | | | | | | |
| ESS score, mean (SD) | 17.3 | (2.8) | 17.3 | (3.5) | 16.9 | (3.7) | 17.2 | (2.8) |
| SBP (mmHg), mean (SD) | 122.6 | (13.6) | 121.1 | (13.3) | 122.0 | (17.1) | 120.2 | (10.2) |
| DBP (mmHg), mean (SD) | 75.3 | (8.7) | 76.0 | (9.0) | 75.9 | (9.9) | 76.2 | (7.0) |

Non-dippers, n (%)

| | | | | | | | | |
|---|---|---|---|---|---|---|---|---|
| SBP | 30 | (60.0) | 22 | (47.8) | 29 | (61.7) | 23 | (54.8) |
| DBP | 17 | (34.0) | 13 | (28.3) | 20 | (42.6) | 14 | (33.3) |
| MAP | 26 | (52.0) | 19 | (41.3) | 27 | (57.4) | 19 | (45.2) |

^{a}Sample size: placebo, n = 58; solriamfetol 75 mg, n = 58; 150 mg, n = 57; 300 mg, n = 59.
BMI, body mass index;
DBP, diastolic blood pressure;
ESS, Epworth Sleepiness Scale;
MAP, mean arterial pressure;
MWT, Maintenance of Wakefulness Test;
SBP, systolic blood pressure;
SD, standard deviation

TABLE 13

| | Solriamfetol | | |
|---|---|---|---|
| Placebo | 75 mg | 150 mg | 300 mg |
| (N = 59) | (N = 59) | (N = 59) | (N = 59) |

SBP (mmHg)

| | | | | |
|---|---|---|---|---|
| n | 50 | 46 | 46 | 41 |
| Mean (SD) | −0.3 (9.28) | 1.8 (6.53) | −0.5 (5.51) | 2.4 (5.97) |

DBP (mmHg)

| | | | | |
|---|---|---|---|---|
| n | 50 | 46 | 46 | 41 |
| Mean (SD) | −0.1 (7.23) | 1.4 (5.13) | 0.4 (4.46) | 3.0 (4.97) |

HR (bpm)

| | | | | |
|---|---|---|---|---|
| n | 50 | 46 | 46 | 41 |
| Mean (SD) | −0.6 (7.00) | 1.0 (7.95) | 0.7 (7.12) | 5.3 (7.55) |

ABPM, ambulatory blood pressure monitoring;
DBP, diastolic blood pressure;
bmp, beats per minute;
HR, heart rate;
mmHg, millimeters mercury;
SBP, systolic blood pressure;
SD, standard deviation

TABLE 14

| | | | Solriamfetol | | | | | | | |
|---|---|---|---|---|---|---|---|---|---|---|
| | Placebo | | 37.5 mg | | 75 mg | | 150 mg | | 300 mg | |
| | (N = 119) | | (N = 58) | | (N = 62) | | (N = 117) | | (N = 118) | |
| Age (years), mean (SD) | 54.1 | (11.4) | 57.1 | (10.2) | 54.4 | (11.5) | 52.7 | (10.6) | 53.2 | (10.6) |
| Male, n (%) | 77 | (64.7) | 39 | (67.2) | 35 | (56.5) | 72 | (61.5) | 74 | (62.7) |

Race, n (%)

| | | | | | | | | | | |
|---|---|---|---|---|---|---|---|---|---|---|
| White | 87 | (73.1) | 45 | (77.6) | 46 | (74.2) | 93 | (79.5) | 90 | (76.3) |
| Black or African | 26 | (21.8) | 10 | (17.2) | 14 | (22.6) | 18 | (15.4) | 21 | (17.8) |
| American | | | | | | | | | | |
| Asian | 4 | (3.4) | 3 | (5.2) | 1 | (1.6) | 3 | (2.6) | 6 | (5.1) |
| BMI (kg/m²), mean (SD) | 33.1 | (5.2) | 34.1 | (5.3) | 33.4 | (5.7) | 33.3 | (4.8) | 32.9 | (5.6) |
| MWT mean sleep latency | 12.4 | (7.2) | 13.6 | (8.1) | 13.1 | (7.2) | 12.5 | (7.2) | 12.0 | (7.3) |
| (min), mean (SD)a | | | | | | | | | | |
| ESS score, mean (SD) | 15.6 | (3.3) | 15.1 | (3.5) | 14.8 | (3.5) | 15.1 | (3.4) | 15.2 | (3.1) |
| AHI, mean (SD)^{b} | 10.9 | (19.2) | 9.8 | (14.7) | 6.1 | (9.1) | 8.1 | (14.2) | 7.4 | (13.9) |

Use of primary OSA therapy, n (%)

| | | | | | | | | | | |
|---|---|---|---|---|---|---|---|---|---|---|
| Adherent^{c} | 83 | (69.7) | 40 | (69.0) | 45 | (72.6) | 80 | (68.4) | 86 | (72.9) |
| Nonadherent | 36 | (30.3) | 18 | (31.0) | 17 | (27.4) | 37 | (31.6) | 32 | (27.1) |
| SBP (mmHg), mean (SD) | 127.7 | (12.1) | 128.2 | (14.2) | 127.1 | (11.4) | 126.8 | (12.8) | 127.8 | (12.2) |
| DBP (mmHg), mean | 78.0 | (8.3) | 77.9 | (9.6) | 78.5 | (9.4) | 78.2 | (7.7) | 78.7 | (7.7) |
| (SD) | | | | | | | | | | |

Non-dippers, n (%)

| | | | | | | | | | | |
|---|---|---|---|---|---|---|---|---|---|---|
| SBP | 62 | (65.3) | 27 | (62.8) | 34 | (65.4) | 71 | (69.6) | 50 | (57.5) |
| DBP | 41 | (43.2) | 20 | (46.5) | 28 | (53.8) | 50 | (49.0) | 37 | (42.5) |
| MAP | 55 | (57.9) | 21 | (48.8) | 28 | (53.8) | 60 | (58.8) | 46 | (52.9) |

aSample size: placebo, n = 114; solriamfetol 37.5 mg, n = 55; 75 mg, n = 61; 150 mg, n = 116; 300 mg, n = 116.
^{b}Sample size: placebo, n = 114; solriamfetol 37.5 mg, n = 56; 75 mg, n = 58; 150 mg, n = 116; 300 mg, n = 113.
^{c}Adherence was defined as PAP use for ≥4 hours per night on ≥70% of nights, use of an oral appliance on ≥70% of nights, or receipt of an effective surgical intervention.
AHI, apnea-hypopnea index;
BMI, body mass index;
DBP, diastolic blood pressure;
ESS, Epworth Sleepiness Scale;
MAP, mean arterial pressure;
MWT, Maintenance of Wakefulness Test;
OSA, obstructive sleep apnea;
PAP, positive airway pressure;
SBP, systolic blood pressure;
SD, standard deviation

TABLE 15

| | Solriamfetol | | | |
|---|---|---|---|---|
| Placebo | 37.5 mg | 75 mg | 150 mg | 300 mg |
| (N = 119) | (N = 58) | (N = 62) | (N = 117) | (N = 118) |

SBP (mmHg)

| | | | | | |
|---|---|---|---|---|---|
| n | 94 | 43 | 51 | 102 | 87 |
| Mean (SD) | −0.8 (7.97) | 0.7 (10.19) | 2.2 (9.82) | −0.5 (8.64) | 2.9 (13.41) |

DBP (mmHg)

| | | | | | |
|---|---|---|---|---|---|
| n | 94 | 43 | 51 | 102 | 87 |
| Mean (SD) | −0.1 (5.57) | 0.8 (6.09) | 0.7 (6.55) | −0.2 (5.10) | 2.5 (8.66) |

HR (bpm)

| | | | | | |
|---|---|---|---|---|---|
| n | 94 | 43 | 51 | 102 | 87 |
| Mean (SD) | −0.5 (6.51) | 0.7 (5.57) | 0.4 (7.70) | 1.5 (6.53) | 1.9 (5.56) |

ABPM, ambulatory blood pressure monitoring;
DBP, diastolic blood pressure;
bpm, beats per minute;
HR, heart rate;
mmHg, millimeters mercury;
SBP, systolic blood pressure;
SD, standard deviation

TABLE 16

| | | Solriamfetol | | | |
|---|---|---|---|---|---|
| | Placebo | 37.5 mg | 75 mg | 150 mg | 300 mg |
| 12-week study, N | 119 | 58 | 62 | 117 | 118 |
| Initiated anti-HTN, n (%) | 0 | 1 (1.7) | 0 | 1 (0.9) | 2 (1.7) |
| OLE, N | — | — | 49 | 144 | 224 |
| Initiated anti-HTN, n (%) | — | — | 3 (6.1) | 2 (1.4) | 3 (1.3) |

Anti-HTN, antihypertensive medication;
OLE, open-label extension

TABLE 17

| | | | | | | | Real-World OSA | | | |
|---|---|---|---|---|---|---|---|---|---|---|
| | 12-Week Study | | | | OLE Study | | Populations | | | |
| | Placebo | | Solriamfetol^{a} | | Solriamfetol^{a} | | Bailly et al^{g} | | Tkacova et al^{h} | |
| | (N = 119) | | (N = 355) | | (N = 417) | | (N = 18,263) | | (N = 11,911) | |
| Age (years), | 54.1 | (11.4) | 53.9 | (10.8) | 55.1 | (10.7) | 59 | [50-67]^{b} | 52 | (12) |
| mean (SD) | | | | | | | | | | |
| Male, % | 64.7 | | 62.0 | | 61.6 | | 73.8 | | 69.6 | |
| BMI (kg/m²), | 33.1 | (5.2) | 33.3 | (5.3) | 33.5 | (5.1) | 31 | [27-36]^{b} | 31.3 | (6.7) |
| mean (SD) | | | | | | | | | | |
| AHI, mean | 10.9 | (19.2) | 7.8 | (13.4) | N/A^{c} | | 35 | [26-51]^{b} | N/A | |
| (SD) | | | | | | | | | | |
| Hypertension, | 50.4 | | 48.7 | | 50.4 | | 46.4 | | 41.4 | |
| % | | | | | | | | | | |
| Hyperlipidemia, | 47.1 | | 47.9 | | 50.6 | | 30.44 | | 20.8^{d} | |
| % | | | | | | | | | | |
| Diabetes | 32.8 | | 29.6 | | 32.9 | | 14.7 | | 9.7^{e} | |
| mellitus, % | | | | | | | | | | |
| SBP (mm Hg), | 126.3 | [103-147] | 124.9 | [95-167] | 128.0 | [96-178] | 130 | [125-140]^{b} | 134 | (18)^{f} |
| median [range] | | | | | | | | | | |
| DBP (mm Hg), | 77.4 | [60-95] | 77.3 | [52-101] | 79.0 | [51-111] | 80 | [70-86]^{b} | 82 | (12)^{f} |

median [range]

| | | | | | | | | |
|---|---|---|---|---|---|---|---|---|
| HR (bpm), | 69.4 | [53-92] | 69.3 | [49-99] | 76.5 | [50-108] | N/A | N/A |

median [range]

^{a}Combined dose groups.
^{b}Median [IQR].
^{c}PSG not performed as part of study procedures.
^{d}Dyslipidemia.
^{e}Type 2.
^{f}Mean (SD).
^{g}Bailly S, et al. PLoS One. 2016; 11(6): e0157318.
^{h}Tkacova R, et al. Eur Respir J. 2014; 44(4): 931-41.
AHI, apnea-hypopnea index;
BMI, body mass index;
DBP, diastolic blood pressure;
HR, heart rate;
IQR, interquartile range;
N/A, not applicable;
OLE, open-label extension,
OSA, obstructive sleep apnea,
PSG, polysomnography;
SBP, systolic blood pressure;
SD, standard deviation

TABLE 18

| | | Solriamfetol | | | | | |
|---|---|---|---|---|---|---|---|
| | Placebo | 37.5 mg | 75 mg | 150 mg | | 300 mg | |
| 12-week study, N | 119 | 58 | 62 | 117 | | 118 | |
| AE of HTN/increased BP, n (%) | 0 | 0 | 0 | 5 | (4.3) | 5 | (4.2) |
| Discontinuation due to AE of | 0 | 0 | 0 | 1 | (0.9)^{b} | 0 | |
| HTN/increased BP, n (%) | | | | | | | |
| OLE, N | — | — | 49 | 144 | | 224 | |
| AE of HTN/increased BP, n (%)^{c} | — | — | 4 (1.0) | 6 | (1.5) | 12 | (3.7) |
| Discontinuation due to AE of | — | — | 1 (0.2)^{d} | 1 | (0.2)^{d} | 1 | (0.3)^{e} |

HTN/increased BP, n (%)

^{b}For this participant, baseline BP was 130/89 mm Hg. On the day of the AE of BP increased (and discontinuation from the study), maximum BP was 191/138 mm Hg; the participant had stopped taking anti-HTN medication ~5 days prior to the AE.
^{c}Percentages are based on number of participants that ever received a given dose level (75 mg, n = 417; 150 mg, n = 402; 300 mg, n = 326).
^{d}Data on BP at time of AE not available for these participants.
^{e}For this participant, parent study baseline BP was 115/70 mm Hg and OLE baseline BP was 134/80 mm Hg. Maximum BP on the day of discontinuation from the study was 158/80 mm Hg.
AE, adverse event;
BP, blood pressure;
HTN, hypertension;
MedDRA, Medical Dictionary for Regulatory Activities;
OLE, open-label extension

1-23. (canceled)

24. A method of ameliorating at least one biomarker of cardiovascular risk in a patient, the method comprising administering (R)-2-amino-3-phenylpropylcarbamate to the patient.

25. The method of claim 24, wherein the patient has a BMI of 25 kg/m² or greater.

26. The method of claim 25, wherein the patient has a BMI in the range from 25 kg/m² to less than 30 kg/m².

27. The method of claim 25, wherein the patient has a BMI of 30 kg/m² or greater.

28. The method of claim 24, wherein the ameliorating at least one biomarker of cardiovascular risk comprises reducing blood pressure level of the patient.

29. The method of claim 28, wherein the reducing blood pressure level is reducing systolic blood pressure.

30. The method of claim 28, wherein the reducing blood pressure is reducing diastolic blood pressure.

31. The method of claim 28, wherein the reducing blood pressure is reducing both systolic blood pressure and diastolic blood pressure.

32. The method of claim 28, wherein blood pressure is reduced by 1 mm Hg or greater.

33. The method of claim 28, wherein blood pressure is reduced by 2 mm Hg or greater.

34. The method of claim 24, wherein the ameliorating at least one biomarker of cardiovascular risk comprises reducing triglycerides level of the patient.

35. The method of claim 34, wherein the ameliorating at least one biomarker of cardiovascular risk further comprises reducing body weight.

36. The method of claim 35, wherein body weight is reduced by 5% or greater.

37. The method of claim 24, wherein the ameliorating at least one biomarker of cardiovascular risk comprises reducing glucose level of the patient.

38. The method of claim 37, wherein the ameliorating at least one biomarker of cardiovascular risk further comprises reducing body weight.

39. The method of claim 38, wherein body weight is reduced by 5% or greater.

40. The method of claim 24, wherein the patient has been receiving (R)-2-amino-3-phenylpropylcarbamate for at least 40 weeks and has over that 40 weeks experienced at least about 5% loss in BMI as compared to before receiving (R)-2-amino-3-phenylpropylcarbamate.

41. The method of claim 24, wherein the patient has been receiving (R)-2-amino-3-phenylpropylcarbamate for at least 40 weeks and has over that 40 weeks experienced at least about 5% loss in body weight as compared to before receiving (R)-2-amino-3-phenylpropylcarbamate.

42. The method of claim 24, wherein the patient does not display an increased propensity toward a non-dipping classification over course of treatment.

43. The method of claim 24, wherein the patient receives about 75-150 mg/day of (R)-2-amino-3-phenylpropylcarbamate.

44. The method of claim 43, wherein the patient receives about 37.5, 75, 150, or 300 mg/day of (R)-2-amino-3-phenylpropylcarbamate.

45. The method of claim 24, wherein the patient receives 75 mg/day of (R)-2-amino-3-phenylpropylcarbamate for at least 3 days followed by 150 mg/day of (R)-2-amino-3-phenylpropylcarbamate.

46. The method of claim 24, wherein the patient does not have narcolepsy.
